(19) United States
(12) Patent Application Publication
(10) Pub. No.: US 2011/0177006 A1
(43) Pub. Date: Jul. 21, 2011

(54) Dithienofuran Dyes for Imaging and Therapy

(76) Inventors: Raghavan Rajagopalan, St. Peters, MO (US); William L. Neumann, St. Louis, MO (US); Amruta Poreddy, St. Louis, MO (US); John N. Freskos, St. Louis, MO (US); Richard B. Dorshow, St. Louis, MO (US)

(21) Appl. No.: 13/121,210

(22) PCT Filed: Sep. 29, 2009
(86) PCT No.: PCT/US2009/058679

§ 371 Date: Mar. 28, 2011

Related U.S. Application Data
(60) Provisional application No. 61/194,600, filed on Sep. 29, 2008.

(51) Int. Cl. A61K 49/00; C07D 409/14; C07D 217/00; C07D 209/02; C07D 401/14; C07D 473/00; C07K 14/00; C07K 2/00; C07H 21/00; C12N 9/00; C07K 16/00; C07H 3/00; C07K 14/575; C07K 9/00; C07D 487/22; C07D 279/18; C09B 56/00; A61P 35/00; A61M 37/00

(52) U.S. Cl. 424/9.61; 549/43; 424/9.6; 546/139; 546/281.1; 548/465; 544/405; 544/264; 530/300; 530/350; 536/23.1; 435/183; 530/387.1; 536/123.1; 530/395; 530/322; 540/145; 544/35; 534/752; 604/20

(57) ABSTRACT

The invention provides optical agents, including compositions, preparations and formulations, and methods of using and making optical agents. Optical agents of the present invention include dyes, and derivatives thereof, having a fused ring backbone structure having dithienofuran core. In some embodiments, dyes of the present invention are dithienofuran dyes having a dithienofuran core optionally functionalized to provide useful optical, biological, pharmacokinetic and/or physical properties.

### CROSS-REFERENCE TO RELATED APPLICATIONS

This application claims the benefit of U.S. Provisional Patent Application No. 61/194,600 filed on Sep. 29, 2008, which is hereby incorporated by reference in its entirety to the extent not inconsistent with the present description.

### BACKGROUND

Optical agents currently play a central role in a large number of in vivo, in vitro and ex vivo clinical procedures including important diagnostic and therapeutic procedures. Photodiagnostic and phototherapeutic agents, for example, include a class of molecules capable of absorbing, emitting, or scattering electromagnetic radiation applied to a biological material, particularly in the visible and near infrared regions of the electromagnetic spectrum. This property of optical agents is used in a range of biomedical applications for visualizing, imaging or otherwise characterizing biological materials and/or achieving a desired therapeutic outcome. Recent developments in targeted administration and delivery of optical agents, and advanced systems and methods for applying and detecting electromagnetic radiation in biological environments has considerably expanded the applicability and effectiveness of optical agents for clinical applications.

Important applications of optical agents that absorb and/or emit in the visible and near-infrared (NIR) region of the electromagnetic spectrum include their use in biomedical imaging and visualization. For example, compounds absorbing and/or emitting light in these regions of the electromagnetic spectrum currently are useful for optical tomography, optoacoustic tomography, optical coherence tomography, confocal scanning laser tomography, optical coherence tomography, and fluorescence endoscopy; techniques which have emerged as essential molecular imaging techniques for imaging and visualizing biological processes at the organ, cellular and subcellular (e.g., molecular) levels. Biomedical images are generated, for example, by detecting electromagnetic radiation, nuclear radiation, acoustic waves, electrical fields, and/or magnetic fields transmitted, emitted and/or scattered by components of a biological sample. Modulation of the energy or intensity of the applied radiation yields patterns of transmitted, scattered and/or emitted radiation, acoustic waves, electrical fields or magnetic fields that contain useful anatomical, physiological, and/or biochemical information. A number of applications of biomedical imaging have matured into robust, widely used clinical techniques including planar projection and tomographic X-ray imaging, magnetic resonance imaging, ultrasound imaging, and gamma ray imaging.

Established optical imaging and visualization techniques are based on monitoring spatial variations in a variety of optical parameters including the intensities, polarization states, and frequencies of transmitted, reflected, and emitted electromagnetic radiation. Given that many biological materials of interest are incompatible with ultraviolet light, research is currently directed to developing and enhancing imaging techniques using visible and near infrared (NIR) radiation (from about 400 nm to about 900 nm). In particular, NIR light (700 nm to 900 nm) is useful for visualizing and imaging deeper regions than visible light because electromagnetic radiation of this wavelength range is capable of substantial penetration (e.g., up to four centimeters) in a range of biological media. Optical imaging and visualization using optical agents has potential to provide a less invasive and safer imaging technology, as compared to X-ray, and other widely used nuclear medicine technologies. Applications of optical imaging for diagnosis and monitoring of the onset, progression and treatment of various disease conditions, including cancer, are well established. (See, e.g., D. A. Benaron and D. K. Stevenson, Optical time-of-flight and absorbance imaging of biologic media, Science, 1993, 259, pp. 1463-1466; R. F. Potter (Series Editor), Medical optical tomography: functional imaging and monitoring, SPIE Optical Engineering Press, Bellingham, 1993; G. J. Tearney et al., In vivo endoscopic optical biopsy with optical coherence tomography, Science, 1997, 276, pp. 2037-2039; B. J. Tromberg et al., Non-invasive measurements of breast tissue optical properties using frequency-domain photon migration, Phil. Trans. Royal Society London B, 1997, 352, pp. 661-668; S. Fantini et al., Assessment of the size, position, and optical properties of breast tumors in vivo by noninvasive optical methods, Appl. Opt., 1998, 37, pp. 1982-1989; A. Pelegrin et al., Photoimmunodiagnosis with antibody-fluorescein conjugates: in vitro and in vivo preclinical studies, J. Cell Pharmacol., 1992, 3, pp. 141-145).

Optical agents for in vivo and in vitro biomedical imaging, anatomical visualization and monitoring organ function are described in International Patent Publication WO2008/108941; U.S. Pat. Nos. 5,672,333; 5,698,397; 6,167,297;6,228,344; 6,748,259; 6,838,074; 7,011,817; 7,128,896, and 7,201,892. In this context, optical imaging agents are commonly used for enhancing signal-to-noise and resolution of optical images and extending these techniques to a wider range of biological settings and media. In addition, use of optical imaging agents having specific molecular recognition and/or tissue targeting functionality has also been demonstrated as effective for identifying, differentiating and characterizing discrete components of a biological sample at the organ, tissue, cellular, and molecular levels. Further, optical agents have been developed as tracers for real time monitoring of physiological function in a patient, including fluorescence-based monitoring of renal function. (See International Patent Publication PCT/US2007/0149478). Given their recognized utility, considerable research continues to be directed toward developing improved optical agents for biomedical imaging and visualization.

In addition to their important role in biomedical imaging and visualization, optical agents capable of absorption in the visible and NIR regions have also been extensively developed for clinical applications for phototherapy. The benefits of phototherapy using optical agents are widely acknowledged as this technique has the potential to provide efficacy comparable to radiotherapy, while entirely avoiding exposure of non-target organs and tissue to harmful ionizing radiation. Photodynamic therapy (PDT), in particular, has been used effectively for localized superficial or endoluminal malignant and premalignant conditions. The clinical efficacy of PDT has also been demonstrated for the treatment of various other diseases, injuries, and disorders, including cardiovascular disorders such as atherosclerosis and vascular restenosis, inflammatory diseases, ophthalmic diseases and dermatological diseases. Visudyne and Photofrin, for example, are two optical agents that have been developed for the treatment of macular degeneration of the eye and for ablation of several types of tumors, respectively. (See, e.g., Schmidt-Drfurth, U.; Bringruber, R.; Hasan, T. Phototherapy in ocular vascular disease. IEEE Journal of Selected Topics in Quantum Electronics 1996, 2, 988-996; Mlkvy, P.; Messmann, H.; Regula, J.; Conio, M.; Pauer, M.; Millson, C. E.; MacRobert, A. J.; Brown, S. G. Phototherapy for gastrointestinal tumors using three photosensitizers—ALA induced PPIX, Photofrin, and MTHPC. A pilot study. Neoplasma 1998, 45, 157-161; Grosjean, P.; Wagieres, G.; Fontolliet, C.; Van Den Bergh, H.; Monnier, P. Clinical phototherapy for superficial cancer in the esophagus and the bronchi: 514 nm compared with 630 nm light irradiation after sensitization with Photofrin II. British Journal of Cancer 1998, 77, 1989-1955; Mitten, D.; Ackroyd, R. Phototherapy of Barrett's oesophagus and oesophageal carcinoma—how I do it. Photodiagnostics and Phototherapy 2006, 3, 96-98; and Li, L.; Luo, R.; Liao, W.; Zhang, M.; Luo, Y.; Miao, J. Clinical study of photofrin phototherapy for the treatment of relapse nasopharyngeal carcinoma. Photodiagnostics and Phototherapy 2006, 3, 266-271; See, Zheng Huang “A Review of Progress in Clinical Photodynamic Therapy”, Technol Cancer Res Treat 2005 June; 4(3): 283-293; “Photodiagnosis And Photodynamic Therapy”, Brown 5, Brown E A, Walker I. The present and future role of photodynamic therapy in cancer treatment. Lancet Oncol. 2004; 5:497-508; Triesscheijn M, Baas P, Schellens J H M. “Photodynamic Therapy in Oncology”; The Oncologist. 2006; 11:1034-1044; and Dougherty T J, Gamer C J, Henderson B W, Jori G, Kessel D, Korbelik M, Moan J, Peng Q. Photodynamic Therapy. J. Natl. Cancer Inst. 1998; 90:899-905).

Phototherapy is carried out by administration and delivery of a photosensitizer to a therapeutic target tissue (e.g., tumor, lesion, organ, etc.) followed by photoactivation of the photosensitizer by exposure to applied electromagnetic radiation. Phototherapeutic procedures require photosensitizers that are relatively chemically inert, and become activated only upon irradiation with light of an appropriate wavelength. Selective tissue injury can be induced with light when photosensitizers bind to the target tissues, either directly or through attachment to a bioactive carrier or targeting moiety. Photosensitizers essentially operate via two different pathways, classified as Types 1 and 2. A primary distinction between these classes of photosensitizers is that the Type 1 process operates via direct energy or electron transfer from the photosensitizer to the cellular components thereby inducing cell death, whereas the Type 2 process involves first the conversion of singlet oxygen from the triplet oxygen found in the cellular environment followed by either direct reaction of singlet oxygen with the cellular components or further generating secondary reactive species (e.g. peroxides, hydroxyl radical, etc.) which will induce cell death.

The Type 1 mechanism proceeds via a multistep process involving activation of the photosensitizer by absorption of electromagnetic radiation followed by direct interaction of the activated photosensitizer, or reactive intermediates derived from the photosensitizer, with the target tissue, for example via energy transfer, electron transfer or reaction with reactive species (e.g., radicals, ions, nitrene, carbene etc.) resulting in tissue damage. The Type 1 mechanism can be schematically represented by the following sequence of reactions:

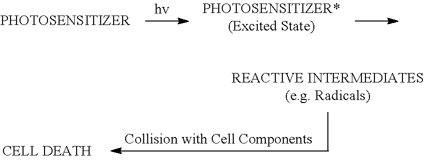

wherein hv indicates applied electromagnetic radiation and (PHOTOSENSITIZER)* indicates excited state of the photosensitizer. The Type 2 mechanism proceeds via a multi-step process involving activation of the photosensitizer by absorption of electromagnetic radiation followed by energy transfer from the activated photosensitizer to oxygen molecules in the environment of the target tissue. This energy transfer process generates excited state oxygen (¹O₂) which subsequently interacts with the target tissue so as to cause tissue damage. The Type 2 mechanism can be schematically represented by the following sequence of reactions:

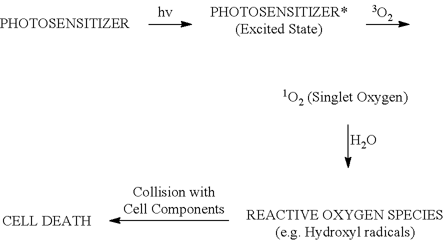

wherein hv indicates applied electromagnetic radiation, (PHOTOSENSITIZER)* indicates photoactivated photosensitizer, ³O₂ is ground state triplet oxygen, and ¹O₂ is excited state singlet oxygen.

The biological basis of tissue injury brought about by tumor phototherapeutic agents has been the subject of intensive study. Various biochemical mechanisms for tissue damage have been postulated, which include the following: a) cancer cells up-regulate the expression of low density lipoprotein (LDL) receptors, and phototherapy (PDT) agents bind to LDL and albumin selectively; (b) porphyrin-like substances are selectively taken up by proliferative neovasculature; (c) tumors often contain increased number of lipid bodies and are thus able to bind to hydrophobic photosensitizers; (d) a combination of “leaky” tumor vasculature and reduced lymphatic drainage causes porphyrin accumulation referred to as “EPR” (enhanced permeability and retention) effect; (e) tumor cells may have increased capabilities for phagocytosis or pinocytosis of porphyrin aggregates; (f) tumor associated macrophages may be largely responsible for the concentration of photosensitizers in tumors; and (g) cancer cells may undergo apoptosis induced by photosensitizers. Among these mechanisms, (f) and (g) are the most general and, of these two alternatives, there is a general consensus that (f) is the most likely mechanism by which the phototherapeutic effect of porphyrin-like compounds is induced.

Much of the research in the past several decades has focused on developing phototherapeutic agents based on the Type 2 (PDT) mechanism. Surprisingly, there has been considerably less attention devoted to Type 1 phototherapeutic agents despite the fact that there are numerous classes of compounds that could potentially be useful for phototherapy that function via this mechanism. Unlike Type 2, the Type 1 process does not require oxygen; and hence Type 1 photosensitizers are expected to be potentially more effective than Type 2 photosensitizers under hypoxic environments typically found in solid tumors. Second, the Type 1 mechanism involves two steps (photoexcitation and direct energy transfer), whereas the Type 2 mechanism involves three steps (photoexcitation, singlet oxygen generation, and energy transfer). Further, studies have recently shown that production of high levels of reactive oxygen species can induce an anti-inflammatory response, which may result in blood vessels to become more “leaky,” thereby increasing the risk of metastasis (Chen, B.; Pogue, B.; Luna, J. M.; Hardman, R. L.; Hoopes, P. J.; Hasan, T. Tumor vascular permeabilization by vascular-targeting photosensitization: effects, mechanism, and therapeutic implications. Clinical Cancer Research 2006, 12(3, Pt.1), 917-923). Targeted Type 1 photosensitizers, by their very nature, are not expected to produce reactive oxygen species; rather, the reactive species produced by these photosensitizers will immediately react with the cellular component at the binding site and trigger cell death. Type 2 phototherapeutic agents, however, do have certain advantages over Type 1 agents. For example, Type 2 agents can potentially be catalytic, i.e., the Type 2 photosensitizer is regenerated once the energy transfer to the oxygen has taken place. In contrast, Type 1 process would generally be expected to require stoichiometric amounts of the photosensitizer in some clinical settings. Table 1 provides a summary of the attributes of Type 1 and Type 2 phototherapeutic agents. Given these attributes, it is clear that development of safe and effective Type 1 phototherapeutic agents would be useful to complement the existing therapeutic approaches provided by Type 2 agents, and to enhance the therapeutic portfolio available for clinicians.

TABLE 1
Comparison between Type 1 and Type 2 processes for phototherapy.

| TYPE 1 PROCESS | TYPE 2 PROCESS |
|---|---|
| Two-step process. | Three-step process. |
| Not well explored. | Very well studied. |
| Light of any wavelength can | Requires red light for optimal |
| be used. | performance. |
| Does not require oxygen. | Requires oxygen. |
| Large classes of compounds. | Limited classes of compounds. |
| Stoichiometric. | Potentially catalytic. |
| Intramolecular energy transfer | Intermolecular energy transfer |
| to generate reactive species. | to generate reactive oxygen species. |
| No products in the market. | Two products are in use. |

Specific optical, chemical and pharmacokinetic properties of optical agents are necessary for their effective use in Type 1 and Type 2 phototherapeutic applications. For example, optical agents for these applications preferably have strong absorption in the visible or NIR regions, and also exhibit low systemic toxicity, low mutagenicity, and rapid clearance from the blood stream. These optical agents must also be compatible with effective administration and delivery to the target tissue, for example by having reasonable solubilities and a low tendency for aggregation in solution. Upon excitation by absorption of visible and NIR electromagnetic radiation, optical agents for Type 1 and 2 phototherapy preferably provide large yields of singlet oxygen (Type 2) or other reactive species, such as free radicals or ions, capable of causing local tissue damage. Both Type 1 and Type 2 photosensitizers typically undergo photoactivation followed by intersystem crossing to their lowest triplet excited state, and therefore, a relatively long triplet lifetime is usually beneficial for providing effective tissue damage. Other useful properties of optical agents for these applications include chemical inertness and stability, insensitivity of optical properties to changes in pH, and compatibility with conjugation to ligands providing targeted delivery via molecular recognition functionality. Multifunctional optical agents have also been developed for phototherapy that are capable of providing both imaging and visual functionality upon excitation at a first range of wavelengths and phototherapeutic functionality upon excitation at a second range of wavelength. (See, U.S. Pat. No. 7,235,685 and International Patent Publication WO 2007/106436).

Optical agents for some phototherapeutic applications preferably exhibit a high degree of selectivity for the target tissue. Selectivity provided by optical agents facilitates effective delivery to a target tissue of interest and provides a means of differentiating different tissue classes during therapy. Selective tissue injury can be induced with light when photosensitizers bind to the target tissues either directly, as in the case of Photofrin, or through attachment to a bioactive carrier, or through in situ biochemical synthesis of the photosensitizer in localized area, as in the case of 2-aminolevulinic acid, which is an intermediate in the biosynthesis of porphyrin. Previous studies have shown that certain dyes selectively localize in tumors and serve as a powerful probe for the detection and treatment of small cancers. (D. A. Belinier et al., Murine pharmacokinetics and antitumor efficacy of the photodynamic sensitizer 2[I-hexyloxyethyl]-2-devinyl pyropheophorbidea, J. Photochem. Photobiol., 1993, 20, pp. 55-61; G. A. Wagnieres et al., In vivo fluorescence spectroscopy and imaging for ontological applications, Photochem. Photobiol., 1998, 68, pp. 603-632; J. S. Reynolds et al., Imaging of spontaneous canine mammary tumors using fluorescent contrast agents, Photochem. Photobiol., 1999, 70, pp. 87-94). It is recognized in some situations, however, that many dyes do not localize preferentially in malignant tissues. A number of strategies have been developed for imparting selectivity and/or targeting functionality by incorporation of a molecular recognition component in the optical agent. For example, targeting of fluorescent dyes to tumors has been demonstrated using dye conjugates with antibodies and peptides for diagnostic imaging of tumors. (See, Achilefu et al., Novel receptor-targeted fluorescent contrast agents for in vivo imaging of tumors, Investigative Radiology, 2000, 35, pp. 479-485; Ballou et al., Tumor labeling in vivo using cyanine conjugated monoclonal antibodies, Cancer Immunology and Immunotherapy, 1995, 41, pp. 257-263; and Licha et al., New contrast agent for optical imaging: acid cleavable conjugates of cyanine dyes with biomolecules, in Biomedical Imaging: Reporters, Dyes and Instrumentation, Proceedings of SPIE, 1999, 3600, pp. 29-35). Therefore, receptor-target mediated phototherapy agents provide a promising pathway for achieving site selective activation at various target tissues.

As will be generally recognized from the foregoing, a need currently exists for optical agents for biomedical applications. Specifically, optical agents for imaging, visualization and phototherapy are needed having enhanced specificity for important target tissue classes, such as tumors and other lesions. In addition, optical agents are needed having enhanced optical, physical, chemical and pharmacokinetic properties for administration, delivery and excitation with electromagnetic radiation.

### SUMMARY

The invention relates generally to optical agents for biomedical applications including imaging, visualization, phototherapy and diagnostic monitoring of cells and tissue. Compounds provided absorb and emit spectral energy in the visible, near infrared, and/or other wavelength ranges useful for optical detection, imaging, monitoring and phototherapy in biomedical procedures. The invention provides optical agents, including compositions, preparations and formulations thereof, and methods of using and making optical agents. The present optical agents enable a versatile diagnostic platform useful for in viva, in vitro and ex viva diagnostic monitoring, visualization and imaging applications, such as, but not limited to, tomographic, photoacoustic and/or sonofluorescent imaging; monitoring and evaluating organ functioning; anatomical visualization; coronary angiography; and fluorescence endoscopy. The optical agents of the invention also enable a versatile phototherapy platform for treatment of a range of pathological conditions, including for the treatment of cancers.

More specifically, optical agents of the present invention include dyes, and derivatives thereof, having a fused ring backbone structure with an dithienofuran core. In some embodiments, dyes of the present invention are fused ring thiophene and furan containing dyes having a dithienofuran core optionally functionalized to provide useful optical, biological, pharmacokinetic and/or physical properties. Optical agents of the present invention further include conjugates, for example, bioconjugates comprising a dithienofuran dye linked to one or more targeting ligands such as a polypeptide, protein, oligonucleotide or other ligand capable of providing molecular recognition and/or targeting functionality. Optical agents of the present invention further include compositions comprising a dithienofuran dye linked to a separate photosensitizer component useful for tandum imaging and phototherapy applications. Dithienofuran dyes of the present invention provide functionality as exogenous optical agents for biomedical and bioanalytical applications including imaging, visualization, diagnostic monitoring, and phototherapy.

In an aspect, the invention provides compounds useful as optical agents for diagnostic, bioanalytical and/or therapeutic methods. In an aspect, the invention provides dithienofuran compounds useful as optical agents in a biomedical procedure, for example, for carrying out a diagnostic, bioanalytical and/or phototherapeutic method. In an embodiments, for example, the present invention provides a compound being of the formula (FX1):

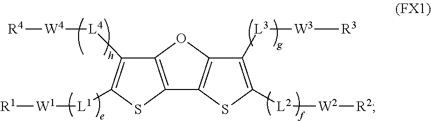

or a pharmaceutically acceptable salt or ester thereof, wherein:

each of L¹, L², L³, and L⁴, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;

each of W¹, W², W³, and W⁴ is independently a single bond, —(CH₂)ₙ—, —(HCCH)ₙ—, —O—, —S—, —SO—, —SO₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, —NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCO(CH₂)n—, —OCOO(CH₂)ₙ—, —CONR²¹(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, —NR²⁶CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, -or —CO(CH₂)ₙNR³³CO—;

each of R¹, R², R³, and R⁴ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₆-C₂₀ alkylaryl, C₁-C₆ alkoxycarbonyl, halo, halomethyl, dihalomethyl, trihalomethyl, —CO₂R⁴⁰—, —SOR⁴¹, —OSR⁴², —SO₂OR⁴³, —CH₂(CH₂OCH₂)_{c}CH₂OH, —PO₃R⁴⁴R⁴⁵, —OR⁴⁶, —SR⁴⁷, —NR⁴⁸R⁴⁹, —NR⁵⁰COR⁵¹, —CH, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —CH(R⁶²)CO₂H, —CH(R⁶³)NH₂, —N₃, PS¹, PS², FL or Bm;

each of a and b is independently an integer selected from the range of 1 to 100;

each of n is independently an integer selected from the range of 1 to 10;

each of e, f, g and h is independently 0 or 1;

each of R¹¹-R³³ is independently hydrogen, C₁-C₂₀ alkyl, or C₅-C₂₀ aryl;

each of R⁴⁰-R⁶¹ is independently hydrogen or C₁-C₁₀ alkyl;

each of R⁶² and R⁶³ is independently a side chain residue of a natural α-amino acid;

each of FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound;

each PS¹ is independently a Type 1 photosensitizer; each PS² is independently a Type 2 photosensitizer; and

each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent.

In an embodiment, for example, the invention provides a compound for use as an optical agent in a phototherapy procedure having formula (FX1), wherein at least one of R¹-R⁴ is PS¹, and optionally at least one of R¹-R⁴ is Bm. In an embodiment, for example, the invention provides compounds having any of formula (FX1)-(FX4), wherein each PS¹ is an azide, azo, diazo, oxaza, or diaza group. In an embodiment, for example, the invention provides a compound for use as an optical agent in a phototherapy procedure having formula (FX1), wherein at least one of R¹-R⁴ is PS², and optionally at least one of R¹-R⁴ is Bm. In an embodiment, for example, the invention provides compounds having any of formula (FX1)-(FX4), wherein each PS² is a group corresponding to a porphyrin, benzoporphyrin, phthalocyanine, phenothiazine, chlorin, bacteriochlorin, phthalocyanine, porphyrin, purpurin, merocyanine, pheophorbides, psoralen, aminolevulinic acid (ALA), hematoporphyrin derivative, porphycene, porphacyanine, cyanine, indocyanine, phthalocyanine, rhodamine, phenoxazine, a phenoselenazine, fluorescein, squaraine, corrin, croconium, azo dye, methine dye, indolenium dye, halogen, anthracyline, C₁-C₂₀ peroxyalkyl, C₁-C₂₀ peroxyaryl, C₁-C₂₀ sulfenatoalkyl, sulfenatoaryl, naphthalocyanine, methylene blue, or chalcogenopyrylium analogue. In an embodiment, for example, the invention provides a compound for use as an optical agent for assessing physiological function of an organ or tissue having formula (FX1), wherein R¹-R⁴ are each a group other than PS¹ or PS². In an embodiment, for example, the invention provides a compound for use as an optical agent for imagining, or visualizing tissue, organs and/or cells having formula (FX1), optionally wherein at least one of R¹-R⁴ is FL. In an embodiment, for example, the invention provides a compound for use as an optical agent for imagining, or visualizing tissue, organs and/or cells having formula (FX1), wherein at least one of R¹-R⁴ is Bm.

As used throughout the present description, reference to embodiments wherein e, f, g and/or h is equal to 0 refers to compounds where L¹, L², L³ or L⁴, respectively, is not present and reference to embodiments wherein e, f, g and/or h is equal to 1 refers to compounds where L¹, L², L³ or L⁴, respectively, is present. For example, W¹ is directly linked to the dithienofuran core when e is equal to 0; and/or W² is directly linked to the dithienofuran core when f is equal to 0; and/or W³ is directly linked to the dithienofuran core when g is equal to 0; and/or W⁴ is directly linked to the dithienofuran core when h is equal to 0. Embodiments wherein W¹ is a single bond and e is equal to 0 refer to compositions having R¹ directly linked to the dithienofuran core. Embodiments wherein W² is a single bond and f is equal to 0 refer to compositions having R² directly linked to the dithienofuran core. Embodiments wherein W³ is a single bond and g is equal to 0 refer to compositions having R³ directly linked to the dithienofuran core. Embodiments wherein W⁴ is a single bond and h is equal to 0 refer to compositions having R⁴ directly linked to the dithienofuran core. As used throughout the present description, the expression “a group corresponding to” an indicated species expressly includes a radical (including a divalent radical), for example an aromatic radical or heterocyclic aromatic radical, of the species or group of species provided in a covalently bonded configuration, optionally with one or more substituents, including but not limited to electron donating groups, electron withdrawing groups, fluorophores, photosensitizers and/or targeting ligands.

Optical agents of this aspect include compounds being of the formula (FX2):

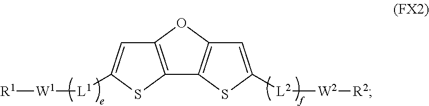

or a pharmaceutically acceptable salt or ester thereof, wherein L¹, L², W¹, W², R¹, R², e, and f are defined as provided in the description of compounds of formula (FX1). Optical agents of this aspect include compounds being of the formula (FX3):

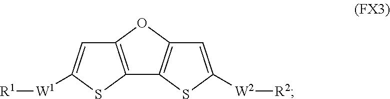

or a pharmaceutically acceptable salt or ester thereof, wherein W¹, W², R¹, and R² are defined as provided in the description of compounds of formula (FX1). Optical agents of this aspect include compounds being of the formula (FX4):

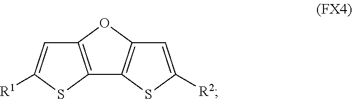

or a pharmaceutically acceptable salt or ester thereof, wherein R¹ and R² are defined as provided in the description of compounds of formula (FX1).

In an embodiment, the invention provides a compound being of the formula (FX2):

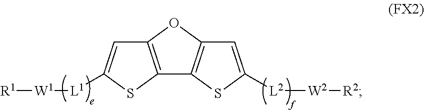

or a pharmaceutically acceptable salt or ester thereof, wherein W¹ is a single bond, —SO—, —SO₂—, or —CO—; and R¹ is —N₃, —SOR⁴¹, or —OSR⁴², and wherein L¹, L², R², W², e, and f are defined as provided in the description of compounds of formula (FX1).

In an embodiment, the invention provides a compound being of the formula (FX3):

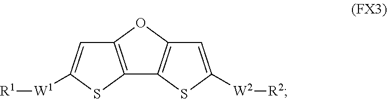

or a pharmaceutically acceptable salt or ester thereof, wherein W¹ is —O—, —S—, —NR¹¹—, —OCO—, —OCOO—, —NR¹³CO—, —CONR¹²—, —OCONR¹⁴—, or —NR¹⁶COO—; W² is —SO—, —SO₂—, —SO₃—, —COO—, or —CONR¹²—; R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm; and R² is hydrogen, C₁-C₂₀ alkyl, C₅-C₂₀ aryl, C₅-C₂₀ alkyaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm. In an embodiment, the invention provides a compound being of formula (FX3), wherein W¹ is —NR¹¹—, or —CONR¹²—; W² is —COO—or —CONR¹²‘; R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm; and R² is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm.

The present invention includes therapeutic agents for biomedical applications comprising purified stereoisomers (e.g., enantiomers and diastereomers), salts (including quarternary salts), and/or ionic forms (e.g., protonated and deprotonated forms) of the compounds of any of formula (FX1)-(FX4), and mixtures thereof. As will be understood by those having general skill in the art, acidic functional groups and basic functional groups of the compounds of any of formula (FX1)-(FX4) may be in protonated or deprotonated states depending on the molecular environment (e.g., pH, ionic strength, composition, etc.), for example during synthesis, formulation and/or administration.

In an embodiment, the invention provides compounds having any of formula (FX1) -(FX4), wherein W¹ is a single bond, —SO—, —SO₂—, or —CO—; and R¹ is —N₃, —SOR⁴¹, or —OSR⁴². In an embodiment, the invention provides compounds having any of formula (FX1)-(FX4), wherein: W¹ is —O—, —S—, —NR¹¹—, —OCO—, —OCOO—, —NR¹³CO—, —CONR¹²—, —OCONR¹⁴—, or —NR¹⁵COO—; W² is —SO—, —SO₂—, —SO₃—, —COO—, or —CONR¹²—; R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm; and R² is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm. In an embodiment, the invention provides compounds having any of formula (FX1)-(FX4), wherein: W¹ is —NR¹¹—, or —CONR¹²—; W² is —COO— or —CONR¹²—; R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL or Bm; and R² is hydrogen, C₁-C₂₀ alkyl, C₅-C₂₀ aryl, C₅-C₂₀ alkylaryl, —CH₂(CHOH)ₐR⁶³, —(CH₂CH₂O)_{b}R⁶¹, —CH(R⁶²)CO₂H, —CH(R⁶³)NH₂, PS¹, PS², FL or Bm. In an embodiment, the invention provides compounds having any of formula (FX1)-(FX4), wherein R³ and R⁴ are each a hydrogen, W³ and W⁴ are each a single bond, and wherein g and h are each 0 (i.e., L³ and L⁴ are not present).

In certain embodiments of the invention, the composition of ring substituents (e.g., R¹-R⁴) on the dithienofuran core in compositions having formula (FX1)-(FX4) is selected to achieve preselected properties, such as optical, physiochemical and pharmacokinetic properties useful for biomedical applications. As used herein, the term dithienofuran core refers to the fused thiophene and furan rings of the present compounds. The invention provides, for example, compositions having any one of (FX1)-(FX4) wherein at least one of R¹-R⁴ is an electron withdrawing group (EWG) bonded directly or indirectly to a carbon atom of the dithienofuran core and at least one of R¹-R⁴ is an electron donating group (EDG) bonded directly or indirectly to a carbon atom of the dithienofuran core. Incorporation of a combination of an EWD and an EDG as substituents of different carbon atoms of the dithienofuran core is particularly beneficial for providing optical agents having large extinction coefficients in the visible and near infrared regions of the electromagnetic spectrum (e.g., 350 nm-1300 nm, optionally 400 nm to 900 nm), emission in the visible and near infrared regions(e.g., 350 nm-1300 nm, optionally 500-900 nm), a large fluorescence quantum yield (e.g., >0.1) and a Stoke's shift useful for optical detection and imaging (e.g., Stoke's shift >10 nm). In some embodiments, for example, an electron withdrawing group and electron donating group are positioned on adjacent carbon atoms of the dithienofuran core. Alternatively, the invention includes embodiments wherein an electron withdrawing group and an electron donating group are positioned on non-adjacent carbon atoms of the dithienofuran core. Multiple electron withdrawing groups and/or electron donating groups on each substituent arm of the dithienofuran core are contemplated by the compositions of this aspect of the invention. By way of example, one EWG arm may comprise two, three, or more electron withdrawing groups bonded to the dithienofuran core via a common linking moiety and/or one EDG arm may comprise two, three, or more electron donating groups bonded to the dithienofuran core via a common linking moiety.

In an embodiment, the present invention provides compositions having any one of formula (FX1)-(FX4), wherein at least one of R¹-R⁴ is C₁-C₂₀ alkyl, —OR⁴⁶, —OR⁴⁷, —NR⁴⁸R⁴⁹, and —NR⁵⁰COR⁵¹, and optionally at least one of R¹-R⁴ is Bm. In an embodiment, the present invention provides compositions having any one of formula (FX1)-(FX4), wherein at least one of R¹-R⁴ is —NR⁴⁸R⁴⁹, or —NR⁶⁰COR⁵¹, and optionally at least one of R¹-R⁴ is Bm. In an embodiment, the present invention provides compositions having any one of formula (FX1)-(FX4), wherein R¹ is —NR⁴⁸R⁴⁹, or R² is —NR⁴⁸R⁴⁹, or R³ is —NR⁴⁸R⁴⁹, or R⁴ is —NR⁴⁸R⁴⁹, and optionally at least one of R¹-R⁴ is Bm. In an embodiment, the present invention provides compositions having any one of formula (FX1)-(FX4), wherein at least one of R¹-R⁴ is —CN, halo, —CO₂R⁴⁰, —COR⁵⁴, —NO₂, —SO₂R⁵⁵, C₁-C₁₀ acyl, or —SO₂NR⁵⁸R⁵⁹, and optionally at least one of R¹-R⁴ is Bm. In an embodiment, the present invention provides compositions having any one of formula (FX1)-(FX4), wherein at least one of R¹-R⁴ is —CN, —CO₂R⁴⁰, or —COR⁵⁴, and optionally at least one of R¹-R⁴ is Bm. In an embodiment, the present invention provides compositions having any one of formula (FX1)-(FX4), wherein R¹ is —CN, or R² is —CN, or R³ is —CN, or R⁴ is —CN, and optionally at least one of R¹-R⁴ is Bm. In an embodiment, the present invention provides compositions having any one of formula (FX1)-(FX4), wherein R1 _{is —CO}₂R⁴⁰, or R² is —CO₂R⁴⁰, or R³ is —CO₂R⁴⁰, or R⁴ is —CO₂R⁴⁰. In an embodiment, the present invention provides compositions having any one of formula (FX1)-(FX4), wherein at least one of R¹-R⁴ is —CO₂R⁴⁰, —COR⁵⁴, —SO₂NR⁵⁸R⁵⁹ or —SO₂R⁵⁵, optionally —CO₂H, —COH, —SO₂NH₂ or —SO₂H. In an embodiment, the present invention provides compositions having any one of formula (FX1)-(FX4), wherein at least one of R¹-R⁴ is a halo group, such as —F, —Cl, —Br or —I, and optionally at least one of R¹-R⁴ is Bm. In an embodiment, the present invention provides compositions having any one of formula (FX1)-(FX4), wherein at least one of R¹-R⁴ is —NR⁴⁸R⁴⁹ or —NR⁵⁰COR⁵¹ and wherein at least one of R¹-R⁴ is —CN, —CO₂R⁴⁰,—COR⁵⁴, —SO₂NR⁵⁸R⁵⁹ or —SO₂R⁵⁵. In an embodiment, the present invention provides compositions having any one of formula (FX1)-(FX4), wherein at least one of R¹-R⁴ is —NR⁴⁸R⁴⁹ and wherein at least one of R¹-R⁴ is —CO₂R⁴⁰, —COR⁵⁴, —SO₂NR⁵⁸R⁵⁹ or —SO₂R⁵⁵. In an embodiment, the present invention provides compositions having any one of formula (FX1)-(FX4), wherein at least one of R¹-R⁴ is —NR⁴⁸R⁴⁹ and wherein at least one of R¹-R⁴ is —CN.

In an embodiment, the invention provides compounds with electron-donating and electron-withdrawing groups attached to adjacent positions of the dithienofuran core. In an embodiment, the invention provides compounds with electron-donating and electron-withdrawing groups attached to non-adjacent positions of the dithienofuran core. In an embodiment, for example, provided are compounds of formula (FX1) to (FX4) wherein:
(a) any one of R¹ and R⁴ is C₁-C₆ alkyl, —OR⁴⁶, —SR⁴⁷, —NR⁴⁸R⁴⁹, or —NR⁵⁰COR⁵¹ and the other of R¹ and R⁴ is —CN, —CO₂R⁴⁰, —SO₂OR⁴³, —CONR⁵²R⁵³, —COR⁴⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹; or
(b) any one of R² and R³ is C₁-C₆ alkyl, —OR⁴⁶, —SR⁴⁷, —NR⁴⁸R⁴⁹, or —NR⁵⁰COR⁵¹ and the other of R² and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹; or
(c) any one of R¹ and R² is C₁-C₆ alkyl, —OR⁴⁶, —SR⁴⁷, —NR⁴⁸R⁴⁹, or —NR⁵⁰COR⁵¹ and the other of R¹ and R² is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹; or
(d) any one of R⁴ and R³ is C₁-C₆ alkyl, —OR⁴⁶, —SR⁴⁷, —NR⁴⁸R⁴⁹,or —NR⁵⁰COR⁵¹ and the other of R⁴ and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹.
   In an embodiment, for example, provided are compounds of formula (FX1) to (FX4) wherein:
(e) any one of R¹ and R³ is C₁-C₆ alkyl, —OR⁴⁶, —SR⁴⁷, —NR⁴⁸R⁴⁹, or —NR⁵⁰COR⁵¹ and the other of R¹ and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁵R⁵⁹; or
f) any one of R¹ and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹ and the other of R¹ and R³ is C₁-C₆ alkyl, —OR⁴⁶, —SR⁴⁷, —NR⁴⁸R⁴⁹, or —NR⁵⁰COR⁵¹; or
(f) any one of R² and R⁴ is C₁-C₆ alkyl, —OR⁴⁶, —SR⁴⁷, —NR⁴⁸R⁴⁹, or —NR⁵⁰COR⁵¹ and the other of R² and R⁴ is —CN, —CO₂R⁴⁰, —SO₂OR⁴³, —CONR⁵²R⁵³, —COR⁵⁴, —NO₂, —SOR⁴¹, —SO₂R⁵⁵, —PO₆R⁵⁶R⁵⁷, halo, C₁-C₆ acyl, trihalomethyl, or —SO₂NR⁵⁸R⁵⁹; or
(g) any one of R² and R⁴ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹ and the other of R² and R⁴ is C₁-C₆ alkyl, —OR⁴⁶, —SR⁴⁷, —NR⁴⁸R⁴⁹, or —NR⁵⁰COR⁵¹.
   In an embodiment, for example, provided are compounds of formula (FX1) to (FX4) wherein:
(h) any two of R¹, R² and R³ is C₁-C₆ alkyl, —OR⁴⁶, —SR⁴⁷, —NR⁴⁸R⁴⁹, or —NR⁵⁰COR⁵¹ and the other of R¹, R² and R³ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹; or
(i) any two of R², R³ and R⁴ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹ and the other of R², R³ and R⁴ is C₁-C₆ alkyl, —OR⁴⁶, —SR⁴⁷, —NR⁴⁸R⁴⁹, or —NR⁵⁰COR⁵¹; or
(j) any two of R¹, R³ and R⁴ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹ and the other of R¹, R³ and R⁴ is C₁-C₆ alkyl, —OR⁴⁶, —SR⁴⁷, —NR⁴⁸R⁴⁹, or —NR⁵⁹COR⁵¹; or
(k) any two of R¹, R² and R⁴ is —CN, —CO₂R⁴⁰, —SO₂OR⁴³, —CONR⁵²R⁵³, —COR⁵⁴, —NO₂, —SOR⁴¹, —SO₂R⁵⁵, —PO₃R⁵⁶R⁵⁷, halo, C₁-C₆ acyl, trihalomethyl, or —SO₂NR⁵⁸R⁵⁹ and the other of R¹, R² and R⁴ is C₁-C₆ alkyl, —OR⁴⁶, —SR⁴⁷, —NR⁴⁸R⁴⁹, or —NR⁵⁰COR⁵¹; or
(l) any two of R¹, R², R³ and R⁴ is —CN, —CO₂R⁴⁰, —SO₂OR⁴³, —CONR⁵²R⁵³, —COR⁵⁴, —NO₂, —SOR⁴¹, —SO₂R⁵⁶, —PO₃R⁵⁶R⁵⁷, halo, C₁-C₆ acyl, trihalomethyl, or —SO₂NR⁵⁸R⁵⁹ and the other two of R¹, R², R³ and R⁴ is C₁-C₆ alkyl, —OR⁴⁶, —SR⁴⁷, —NR⁴⁸R⁴⁹, or —NR⁵⁰COR⁵¹.

In an embodiment, the invention provides optical agents for phototherapy having a targeting ligand or other molecular recognition component for delivering the optical agent to a selected organ, tissue, or other cell material. Incorporation of a targeting ligand or molecular recognition component in some compounds and methods of the invention enables targeted delivery such that at least a portion of phototherapeutic agent administered to a subject accumulates at a preselected, desired site, such as the site of an organ, tissue, tumor or other lesion, prior to or during exposure to electromagnetic radiation. Targeting ligands of the present invention may be covalently bonded to, or non-covalently associated with, the dithienofuran core structure of formulae (FX1)-(FX4). The invention includes, for example, compounds of any one of formula (FX1)-(FX4), wherein at least one of R¹-R⁴ is independently a targeting ligand (abbreviated as “Bm” throughout this description). In an embodiment, for example, the invention includes compounds wherein R¹ is Bm and W¹ is —NR¹³CO—, —CONR¹²—OCONR¹⁴—, —NR¹⁵COO—, or —NR¹⁶CONR¹⁷—; or R² is Bm and W² is —NR¹³CO—, —CONR¹²—OCONR¹⁴—, —NR¹⁵COO—, or —NR¹⁶CONR¹⁷—; or R³ is Bm and W³ is —NR¹³CO—, —CONR¹²—OCONR¹⁴—, —NR¹⁵COO—, or —NR¹⁶CONR¹⁷—; or R⁴ is Bm and W⁴ is —NR¹³CO—, —CONR¹²—OCONR¹⁴—, —NR¹⁵COO—, or —NR¹⁶CONR¹⁷—. In an embodiment, for example, invention includes, for example, compounds of any one of formula (FX1)-(FX4), wherein at least one of R¹-R⁴ is independently a polypeptide comprising 2 to 30 amino acid units. In an embodiment, for example, invention includes, for example, compounds of any one of formula (FX1)-(FX4), wherein at least one of R¹-R⁴ is independently an antibody or fragment thereof. In an embodiment, for example, invention includes, for example, compounds of any one of formula (FX1)-(FX4), wherein at least one of R¹-R⁴ is independently a polynucleotide comprising 1 to 50 nucleic acid units.

Compounds of the invention optionally include a photosensitizer component that generates reactive species (e.g., radicals, nitrenes, carbenes, ions, and/or singlet oxygen) upon absorption of electromagnetic radiation. In an embodiment, for example, the invention includes compounds having any one of formula (FX1)-(FX4), wherein at least one of at least one of R¹-R⁴ is independently a Type 1 photosensitizer. In an embodiment, for example, the invention includes compounds having any one of formula (FX1)-(FX4), wherein at least one of R¹-R⁴ is independently a Type 2 photosensitizer. In an embodiment, for example, invention includes compounds of any one of formula (FX1)-(FX4), wherein at least one of R¹-R⁴ is an azide group (—N₃), and optionally at least one of R¹-R⁴ is Bm, wherein optionally exposure to electromagnetic radiation results in cleavage of one or more photolabile nitrogen-nitrogen bonds and/or nitrogen-carbon bond, thereby generating reactive species such as radicals, ions, nitrene, or carbene. In an embodiment, for example, invention includes, for example, compounds of any one of formula (FX1)-(FX4), wherein at least one of R¹-R⁴ is an azo group, and optionally at least one of R¹-R⁴ is Bm, wherein optionally exposure to electromagnetic radiation results in cleavage of one or more photolabile nitrogen-nitrogen bond and/or nitrogen-carbon bond, thereby generating reactive species such as radicals, ions, nitrene, or carbene. In an embodiment, for example, invention includes, for example, compounds of any one of formula (FX1)-(FX4), wherein at least one of R¹-R⁴ is a diazo group, and optionally at least one of R¹-R⁴ is Bm, wherein optionally exposure to electromagnetic radiation results in cleavage of one or more photolabile nitrogen-nitrogen bond and/or nitrogen-carbon bond, thereby generating reactive species such as radicals, ions, nitrene, or carbene. In an embodiment, for example, invention includes, for example, compounds of any one of formula (FX1)-(FX4), wherein at least one of R¹-R⁴ is an oxaza group, and optionally at least one of R¹-R⁴ is Bm, wherein optionally exposure to electromagnetic radiation results in cleavage of one or more photolabile nitrogen-oxygen bond, oxygen-carbon bond and/or nitrogen-carbon bond, thereby generating reactive species such as radicals, ions, nitrene, or carbene. In an embodiment, for example, invention includes, for example, compounds of any one of formula (FX1)-(FX4), wherein at least one of R¹-R⁴ is an diaza group, and optionally at least one of R¹-R⁴ is Bm, wherein optionally exposure to electromagnetic radiation results in cleavage of one or more photolabile nitrogen-nitrogen bond and/or nitrogen-carbon bond, thereby generating reactive species such as radicals, ions, nitrene, or carbene.

In an embodiment, the invention provides compounds of any one of formulae (FX1)-(FX4), wherein each of R¹¹-R³³ is independently hydrogen or a C₁-C₁₀ alkyl, and optionally wherein each of R¹¹-R³³ is hydrogen or a C₁-C₅ alkyl, and optionally wherein each of R¹¹-R³³ is hydrogen. In an embodiment, the invention provides compounds of any one of formulae (FX1)-(FX4), wherein each of R⁴⁰-R⁶¹ is independently hydrogen or C₁-C₅ alkyl. In an embodiment, the invention provides compounds of any one of formulae (FX1)-(FX4), wherein each of R⁴⁰-R⁶¹ is hydrogen.

L¹-L⁴ and W¹-W⁴ groups may be spacer and attaching groups, respectively, for providing an appropriate linkage between R¹-R⁴ and the central dithienofuran core of the compounds of (FX1)-(FX4). In some embodiments, the invention provides compounds of any one of formulae (FX1)-(FX4), wherein any one of L¹-L⁴ is independently a spacer moiety for establishing a steric environment between R¹-R⁴ and the central dithienofuran core providing useful optical, pharmacokinetic, or targeting properties. In some embodiments, the invention provides compounds of any one of formulae (FX1)-(FX4), wherein any one of W¹-W⁴ is independently an attaching moiety for attaching R¹-R⁴ directly or indirectly to the central dithienofuran core. In an embodiment, at least one of L¹-L⁴ is independently —(CH₂)ₘ—, —(HCCH)ₘ—, —(CHOH)ₘ—, or —(CH₂CH₂O)ₘ—, wherein each of m is independently an integer selected from the range of 1 to 100, optionally selected from the range of 1 to 10. In an embodiment, the invention provides compounds of any one of formulae (FX1)-(FX4), wherein at least one of W¹-W⁴ is independently a single bond, —O—, —CO—, —COO—, —OCO—, —OCOO—, —NR¹¹—, —CONR¹²—, —NR¹³CO—; —NR¹⁶CONR¹⁷—, or —NR¹⁸CSNR¹⁹—. In an embodiment, the invention provides compounds of any one of formulae (FX1)-(FX4), wherein at least one of: L¹ and W¹; L² and W²; L³ and W³; and L⁴ and W⁴ combine to form: —(CH₂)ⱼ—, —O(CH₂)ⱼ—, —CO(C₂)ⱼ—, —OCO(CH₂)ⱼ—, —COO(CH₂)ⱼ—, —OCOO(CH₂)ⱼ—, —N(R¹¹)(CH²)ⱼ—, —CON(R¹²)(CH₂)ⱼ—, —N(R¹³)CO(CH₂)ⱼ—, —OCONR¹⁴(CH₂)ⱼ—, —NR¹⁵COO(CH₂)ⱼ—, —NR¹⁶CONR¹⁷(CH₂)ⱼ—, or —NR¹⁸CSNH¹⁹(CH₂)ⱼ—, wherein each j is independently an integer selected from the range of 1 to 100.

In some embodiments, compounds of the invention may optionally include a poly(ethylene glycol) (abbreviated as PEG) component. In an embodiment, for example, the invention provides a composition having any one of the formula (FX1)-(FX4), wherein at least one of L¹-L⁴, and R¹-R⁴ is independently a substituent comprising —(CH₂OCH₂)_{b}—, or a derivative thereof, wherein b is an integer is selected from the range of 1 to 100. Incorporation of a poly(ethylene glycol) glycol component in some compositions of the invention provides pharmacokinetic, chemical, and/or physical properties useful for bioanalytical, diagnostic and/or therapeutic applications. Poly(ethylene glycol) containing compounds of some embodiments of the present invention, for example, provide enhanced biocompatibility, low toxicity and suppress immune responses upon administration. Poly(ethylene glycol) containing compounds of some embodiments of the invention facilitate formulation, administration and/or delivery, for example, by enhancing solubility.

The invention further provides a compound having any one of formula (FX1)-(FX4), or a pharmaceutical formulation thereof, for use in an optical imaging, diagnostic, and/or phototherapeutic biomedical procedure. In an embodiment, the invention provides an optical agent comprising a pharmaceutically acceptable formulation, wherein at least one active ingredient of the formulation is a compound having any one of formula (FX1)-(FX4) provided in a therapeutically effective amount. The invention includes, for example, formulations comprising a compound having any one of formula (FX1)-(FX4) and one or more pharmaceutically acceptable carriers or excipients. In an embodiment, the invention provides a pharmaceutically acceptable formulation for combination therapy comprising a compound having any one of formula (FX1)-(FX4) and one or more additional diagnostic and/or therapeutic agents, such as anti-cancer agents, anti-inflammatory agents, and/or imaging agents (e.g., optical and/or non-optical imaging agents).

In an embodiment, the biomedical procedure comprises: (i) administering (e.g., via intravenous or intraarterial injection, oral administration, topical administration, subcutaneous administration, etc.) to a subject a therapeutically or diagnostically effective amount of the compound having any one of formula (FX1)-(FX4) under conditions sufficient for contacting the compound with a target tissue or cell, wherein the compound selectively binds to or otherwise associates with the target tissue or cell; and optionally (ii) exposing the administered compound to a therapeutically or diagnostically effective amount of electromagnetic radiation. In an embodiment, the biomedical procedure comprises administering or otherwise targeting the administered compound to a target tissue or cell of the subject, such as a tumor, lesion, site of inflammation, vasculature tissue, or an organ. In an embodiment, for example, the target tissue is a tissue type selected from the group consisting of colon, prostate, gastric, esophageal, uterine, endometrial, pancreatic tissue. In an embodiment, the biomedical procedure comprises: (i) administering into a bodily fluid of a subject a diagnostically effective amount of a detectable agent comprising a compound having any one of formula (FX1)-(FX4), wherein the detectable agent is differentially separated from the bodily fluid by the organ or tissue; (ii) exposing the detectable agent in the bodily fluid to electromagnetic radiation for exciting emission from the detectable agent; (iii) measuring the emission from the detectable agent that is in the bodily fluid; and (iv) determining the physiological function of the organ or tissue of the subject based on measurement of the emission.

In an embodiment, the administered compound is exposed at the site of the target tissue or cell to electromagnetic radiation having wavelengths selected over a range of 350 nanometers to 1300 nanometers, optionally having wavelengths selected over a range of 350 nanometers to 900 nanometers. In an embodiment, exposing the administered compound to electromagnetic radiation generates fluorescence, wherein the biomedical procedure further comprises detecting fluorescence from the administered compound. In an embodiment, exposing the administered compound to electromagnetic radiation generates a diagnostically effective amount of fluorescence, for example an amount of fluorescence allowing for optical detection, visualization and/or imaging of the target tissue or an amount providing a detectable signal useful for monitoring organ function in a subject. In an embodiment, a method of the invention further comprises exposing the administered compound at the target tissue to electromagnetic radiation having sufficient power, fluence, intensity and/or dose (net number of photons provided to the target tissue) to provide optical detection, visualization and/or imaging of the target tissue. In an embodiment, a method of the invention further comprises generating image of the fluorescence from the compound. In an embodiment, a method of the invention further comprises visualizing the fluorescence from the compound. In an embodiment, a method of the invention further comprises exciting and measuring fluorescence from the optical agent administered to a bodily fluid of the subject as a function of time, for example, so as to generate a temporal profile of fluorescence useful for characterizing organ function in a subject.

The present invention also provides methods of making and using optical agents, including compounds of formulas (FX1)-(FX4). Methods of this aspect of the present invention include in vivo, in vitro and ex vivo methods for biomedical and bioanalytical applications. For example, provided is a method for assessing physiological function of an organ or tissue using the optical agents of the present invention. In some methods of assessing physiological function, the organ or tissue is a kidney, or tissue or cells thereof, or alternatively the organ or tissue is a liver, or tissue or cells thereof. Methods of the present invention include photodiagnostic and phototherapeutic methods, such as optical imaging, anatomical visualization, endoscopic visualization, image guided surgery, and Type 1 and Type 2 phototherapy of tumors and other lesions. For some compounds for use in vivo, in vitro or ex vivo for imagining or visualizing, the tissue, organs and/or cells is a tumor, tumor site, or other lesion.

The invention further provides a compound having any one of formula (FX1)-(FX4), or a pharmaceutical formulation thereof, for use in a medical phototherapy procedure, such as a Type 1 or Type 2 phototherapy procedure. In an embodiment of this aspect, a compound of the invention has any one of formula (FX1)-(FX4), wherein at least one of R¹-R⁴ is PS¹ or PS². In an embodiment, the medical phototherapy procedure comprises: (i) administering to a subject in need of treatment a therapeutically effective amount of the compound having any one of formula (FX1)-(FX4); and (ii) exposing the administered compound to electromagnetic radiation. In an embodiment, the administered compound is exposed to electromagnetic radiation having wavelengths selected over a range of 350 nanometers to 1300 nanometers, optionally having wavelengths selected over a range of 350 nanometers to 900 nanometers. In an embodiment, exposing the administered compound to electromagnetic radiation generates one or more radicals, nitrenes, carbenes, ions, and/or singlet oxygen. In an embodiment, exposing the administered compound to electromagnetic radiation generates a therapeutically effective amount of photoactivated compound. In an embodiment, exposing the administered compound to electromagnetic radiation generates a therapeutically effective amount of reactive species causing localized cell death or injury. In an embodiment, the medical phototherapy procedure comprises administering, contacting or otherwise targeting the administered compound to a target tissue of the subject, such as a tumor, lesion, site of inflammation, vasculature tissue, or organ. In an embodiment, methods of the invention further comprises exposing the administered compound at the target tissue to light having sufficient power, fluence, intensity and/or dose (net number of photons provided to the target tissue) to result in injury, inactivation and/or death to cells at the target tissue.

In a method, the electromagnetic radiation exposed to the compound of any one of formulae (FX1)-(FX4) does not have wavelengths in the X-ray region of the electromagnetic spectrum. In a method, the electromagnetic radiation exposed to the compound of any one of formulae (FX1)-(FX4) does not have wavelengths in the ultraviolet region of the electromagnetic spectrum. In an embodiment, non-ionizing electromagnetic radiation is used in the present methods. “Non-ionizing electromagnetic radiation” herein refers to electromagnetic radiation wherein a single photon does not have enough energy to completely remove at least one electron from an atom or molecule of the subject's body.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE FIGURES

FIG. 1A provides a chemical formula for a class of dithienofuran dyes having a combination of electron withdrawing group(s) and electron donating group(s) bonded directly or indirectly to the fused ring backbone.

FIG. 1B provides chemical formulae showing examples of specific arrangements and positions of electron withdrawing and electron donating groups useful in certain applications of the present invention.

FIG. 2A provides Scheme 1 for synthesizing exemplary dithienofuran dyes of the present invention with “push-pull” electron donating and electron withdrawing groups.

FIG. 2B provides Scheme 2 and Scheme 3 for synthesizing exemplary dithienofuran compounds of the present invention having a photosensitizer component.

FIG. 2C provides Scheme 4, and Scheme 5 for synthesizing exemplary dithienofuran bioconjugates of the present invention having a ligand component for targeting.

### DETAILED DESCRIPTION

Referring to the drawings, like numerals indicate like elements and the same number appearing in more than one drawing refers to the same element. In general the terms and phrases used herein have their art-recognized meaning, which can be found by reference to standard texts, journal references and contexts known to those skilled in the art. The following definitions are provided to clarify their specific use in the context of the invention.

“Optical agent” generally refers to compositions, preparations, and/or formulations that absorb, emit, or scatter electromagnetic radiation of wavelength, generally in the range of 350-1300 nanometers, within a biologically relevant environment or condition. In some embodiments, optical agents of the present invention, when excited by electromagnetic radiation, undergo emission via fluorescence or phosphorescence pathways. These pathways are useful for diagnostic imaging, visualization, or organ function monitoring. Compounds belonging to this class are commonly referred to as ‘optical imaging agents’ or ‘optical contrast agents.’ In some other embodiments, optical agents of the present invention absorb electromagnetic radiation and undergo photochemical reactions such as photofragmentation of one or more photolabile bonds to generate reactive intermediates such as nitrenes, carbene, free radicals, or ions. This process is useful for a wide range of phototherapy applications, for example in the treatment of tumors or other lesions. Compounds belonging to this class are commonly referred to as ‘photosensitizers.’ The term “photosensitizer” refers to a phototherapeutic agent or a component thereof providing for photoactivation, for example, photoactivation resulting in generation of reactive species (e.g., radicals, ions, nitrene, carbene, excited species, etc.). Photosensitizers of some embodiments undergo photoactivation that initiates bond cleavage reactions, such as photolysis and/or nitrogen extrusion reactions, thereby generating reactive species capable of causing localized cell death or injury. Optical agents include Type 1 and Type 2 phototherapeutic agents.

Compounds and compositions of the invention provide optical agents including photosensitizers, phototherapeutic agents, contrast agents, imaging agents, dyes, and detectable agents; and conjugates, complexes, and derivatives thereof. Optical agents of the present invention include fused ring thiophene and furan containing dyes, and derivatives thereof, having a fused ring dithienofuran core. Optical agents of the present invention include dithienofuran dyes that undergo bond cleavage reactions upon exposure to electromagnetic radiation having wavelengths selected over the range of 350 to 1300 nm, optionally 350-900 nm. Some optical agents of the present invention provide detectable agents that can be administered to a subject and subsequently detected using a variety of optical techniques, including optical imaging, visualization, and one-, two-, three- and point optical detection.

Optical agents include, but are not limited to, phototherapeutic agents (Type 1 and 2), photosensitizers, contrast agents, imaging agents, dyes, detectable agents, photosensitizer agents, photoactivators, and photoreactive agents; and conjugates, complexes, and derivatives thereof.

“Phototherapy procedure” refers to a therapeutic procedure involving administration of a phototherapeutic agent to a patient followed by subsequent excitation by exposure to applied electromagnetic radiation, such as electromagnetic radiation having wavelengths in the visible and/or near IR region of the electromagnetic spectrum such as wavelengths in the range of 350-1300 nanometers, so as to generate a therapeutically effective amount of excited phototherapeutic agent. Phototherapy includes, but is not limited to, photodynamic therapy. As used herein phototherapy includes procedures involving administration of Type 1 and/or Type 2 phototherapeutic agents, optionally further including administration of one or more additional therapeutic agents.

As used herein, “targeting ligand” (abbreviated as Bm) refers to a chemical group and/or substituent having functionality for targeting a compound of any one of formula (FX1)-(FX4) to an anatomical and/or physiological site of a patient, such as a selected cells, tissue or organ. For some embodiments, a targeting ligand is characterized as a ligand that selectively or preferentially binds to a specific biological site(s) (e.g., enzymes, receptors, etc.) and/or biological surface(s) (e.g., membranes, fibrous networks, etc.). In an embodiment, the invention provides compounds having any one or formula (FX1)-(FX4), wherein Bm is amino acid, or a polypeptide comprising 2 to 30 amino acid units. In an embodiment, the invention provides compounds having any one of formula (FX1)-(FX4), wherein Bm is a mono- or polysaccharide comprising 1 to 50 carbohydrate units. In an embodiment, the invention provides compounds having any one or formula (FX1)-(FX4), wherein Bm is a mono-, oligo- or poly-nucleotide comprising 1 to 50 nucleic acid units. In an embodiment, the invention provides compounds having any one or formula (FX1)-(FX4), wherein Bm is a protein, an enzyme, a carbohydrate, a peptidomimetic, a glycomimetic, a glycopeptide, a glycoprotein, a lipid, an antibody, or fragment thereof. In an embodiment, the invention provides compounds having any one or formula (FX1)-(FX4), wherein Bm is a drug, a hormone, or a receptor. In some embodiments, each occurrence of Bm in the compounds of (FX1)-(FX4) is independently a monoclonal antibody, a polyclonal antibody, a metal complex, an albumin, or an inclusion compound such as a cyclodextrin. In some embodiments, each occurrence of Bm in the compounds of (FX1)-(FX4) is independently integrin, selectin, vascular endothelial growth factor, fibrin, tissue plasminogen, thrombin, LDL, HDL, Sialyl LewisX or a mimic thereof, or an atherosclerotic plaque binding molecule. Specific examples of targeting ligands include steroid hormones for the treatment of breast and prostate lesions, whole or fragmented somatostatin, bombesin, and neurotensin receptor binding molecules for the treatment of neuroendocrine tumors, whole or fragmented cholecystekinin receptor binding molecules for the treatment of lung cancer, whole or fragmented heat sensitive bacterioendotoxin (ST) receptor and carcinoembryonic antigen (CEA) binding molecules for the treatment of colorectal cancer, dihydroxyindolecarboxylic acid and other melanin producing biosynthetic intermediates for melanoma, whole or fragmented integrin receptor and atherosclerotic plaque binding molecules for the treatment of vascular diseases, and whole or fragmented amyloid plaque binding molecules for the treatment of brain lesions. In some embodiments, Bm, if present, is selected from heat-sensitive bacterioendotoxin receptor binding peptide, carcinoembryonic antigen antibody (anti-CEA), bombesin receptor binding peptide, neurotensin receptor binding peptide, cholecystekinin receptor binding peptide, somastatin receptor binding peptide, ST receptor binding peptide, neurotensin receptor binding peptide, steriod receptor binding peptide, carbohydrate receptor binding peptide or estrogen. Examples of targeting ligands for specific biomedical applications include steroid hormones for the treatment of breast and prostate lesions, whole or fragmented somatostatin, bombesin, and neurotensin receptor binding molecules for the treatment of neuroendocrine tumors, whole or fragmented cholecystekinin receptor binding molecules for the treatment of lung cancer, whole or fragmented heat stable bacterioenterotoxin (ST) receptor and carcinoembryonic antigen (CEA) binding molecules for the treatment of colorectal cancer, dihyroxyindoiecarboxylic acid and other melanin producing biosynthetic intermediates for melanoma, whole or fragmented integrin receptor and atherosclerotic plaque binding molecules for the treatment of vascular diseases, and whole or fragmented amyloid plaque binding molecules for the treatment of brain lesions. In some embodiments, Bm, if present, is selected from octreotide and octreotate peptides.

“Target tissue” refers to tissue of a subject to which an optical agent is administered or otherwise contacted, for example during a biomedical procedure such as an optical imaging, phototherapy or visualization procedure. Target tissue may be contacted with an optical agent of the invention under in vivo conditions or ex viva conditions. Target tissues in some methods of the invention include cancerous tissue, cancer cells, precancerous tissue, a tumor, a lesion, a site of inflammation, or vasculature tissue. Target tissue in some methods of the invention includes a melanoma cell, a breast lesion, a prostate lesion, a lung cancer cell, a colorectal cancer cell, an atherosclerotic plaque, a brain lesion, a blood vessel lesion, a lung lesion, a heart lesion, a throat lesion, an ear lesion, a rectal lesion, a bladder lesion, a stomach lesion, an intestinal lesion, an esophagus lesion, a liver lesion, a pancreatic lesion, and a solid tumor. Target tissue in some embodiments refers to a selected organ of the subject or component thereof, such as lung, heart, brain, stomach, liver, kidneys, gallbladder, pancreas, intestines, rectum, skin, prostate, ovaries, breast, bladder, blood vessel, throat, ear, or esophagus.

As used herein, “spacer moiety” refers to a component provided between the central dithienofuran core of some compounds of the invention and any of R¹-R⁴. In some embodiments, any one of L¹-L⁴ in formulae (FX1)-(FX4) is a spacer moiety. Spacer moieties useful for some embodiments are provided between any of R¹-R⁴ and the dithienofuran core to enhance the overall chemical, optical, physical and/or pharmacokinetic properties of an optical agent of the present invention. Useful spacer moieties for compounds of the invention having formulae (FX1)-(FX4) include C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—, wherein each of a and b is independently selected from the range of 1 to 100, optionally selected from the range of 1 to 30 and optionally selected from the range of 1 to 10. The invention includes compounds having formulae (FX1)-(FX4), that do not have a spacer moiety.

As used herein, “attaching moiety” refers to a component provided to attach any of R¹-R⁴ directly or indirectly to the dithienofuran core in compounds of the invention. In some embodiments, any one of W¹-W⁴ in formulae (FX1)-(FX4) is an attaching moiety. Attaching moieties may connect to the dithienofuran core directly or may connect to the dithienofuran core via a spacer moiety. Attaching moieties in some embodiments provide a means of derivatizing the dithienofuran core so as to provide optical agents having useful overall chemical optical, physical and/or pharmacokinetic properties, including targeting and molecular recognition functionality. Attaching moieties useful in the present invention include, but are not limited to, a single bond, —(CH₂)ₙ—, —(CHHC)ₙ—, —O—, —S—, —SO—₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, —NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCOO(CH₂)ₙ—, —CONR²¹(CH₂)ₙ—, —CONR²²(CH₂)ₙ—, —NR²³CO(CH₂)ₙ, —OCONR²⁴(CH₂)ₙ—, —NR²⁵COO(CH₂)ₙ—, —NR²⁶CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, -or —CO(CH₂)ₙNR³³CO—, wherein each n is independently selected from the range of 1 to 10.

As used herein, an “electron withdrawing group” (abbreviated as “EWG”) refers to a chemical group that draws electrons or electron density from a center, such as the fused ring backbone structure of a dithienofuran dye of the present invention. In some embodiments, the electron withdrawing group(s) are independently selected from cyano (—CN), carbonyl (—CO), carboxylates (—CO₂R¹), halo (—F, —Cl, —Br, —I), carbamates (—CONR⁵⁵R⁵⁶), acyl (—COR⁵⁷), nitro (—NO₂), sulfinyl (—SOR⁵⁸), sulfonyl (—SO₂R⁵⁹), —SO₂OR⁶⁰, and —PO₃R⁶¹R⁶²; wherein in the context of this description, R⁵⁵-R⁶² are independently selected to enhance biological and/or physiochemical properties of the optical agents of the present invention. In some instances, R⁵⁵-R⁶² are independently selected from any one of a hydrogen atom, an anionic functional group (e.g., carboxylate, sulfonate, sulfate, phosphonate and phosphate) and a hydrophilic functional group (e.g., hydroxyl, carboxyl, sulfonyl, sulfonato and phosphonato). In other instances, R⁵⁵-R⁶² are independently selected form hydrogen, C₁₋₁₀ alkyl, aryl, heteroaryl, —(CH₂)ₐOH, —(CH₂)ₐCO₂H, —(CH₂)ₐSO₃H, —(CH₂)ₐSO₃⁻, —(CH₂)ₐOSO₃H, —(CH₂)ₐOSO₃⁻; —(CH₂)ₐNHSO₃H, —(CH₂)ₐNHSO₃⁻, —(CH₂)ₐPO₃H₂, v(CH₂)ₐPO₃H⁻, —(CH₂)ₐPO₃⁼, —(CH₂)ₐOPO₃H₂, —(CH₂)ₐOPO₃H⁻ and —(CH₂)ₐOPO₃ where a is an integer from 1 to 10. In one example of this embodiment, the EWG(s) are independently selected from —CN, halo, C₁-C₁₀ acyl, —CO₂R⁴⁰, —SOR⁴¹, —OSR⁴², —SO₂OR⁴³, —CONR⁵²R⁵³; —COR⁵⁴; —NO₂, —SO₂R⁵⁵, —SO₂NR⁵⁸R⁵⁹, and —PO₃R⁵⁶R⁵⁷, wherein R⁴⁰-R⁵⁹ are as described in the context of compounds of formulae (FX1). In an embodiment, an EWG is located at the terminus of a substituent arm of the dithienofuran core of the present compounds.

As used herein, an “electron donating group” (abbreviated as “EDG”) refers to a chemical group that releases electrons or electron density to a center, such as the fused ring backbone structure of a dithienofuran dye of the present invention. In some embodiments, the electron donating group(s) are independently selected from C₁-C₁₀ alkyl, C₅-C₁₀ aryl, —(CH₂)ₓOH, —OR⁶⁵, —SR⁶⁶, —NR⁶⁷_{R}⁶⁸, —N(R⁶⁹)COR⁷⁰, and —P(R⁷¹); wherein in the context of this description, R⁶⁵-R⁷¹ are independently selected to enhance biological and/or physiochemical properties of the optical agents of the present invention and wherein x is selected from the range of 1 to 10. In some instances, R⁶⁵-R⁷¹ are independently selected from any one of a hydrogen atom, an anionic functional group (e.g., carboxylate, sulfonate, sulfate, phosphonate and phosphate) and a hydrophilic functional group (e.g., hydroxyl, carboxyl, sulfonyl, sulfonato and phosphonato). In other instances, R⁶⁵-R⁷¹ are independently selected from hydrogen, C₁₋₁₀alkyl, aryl, heteroaryl, —(CH₂)ₐOH, —(CH₂)ₐCO₂H, —(CH₂)ₐSO₃H, —(CH₂)ₐSO₃⁻, —(CH₂)ₐOSO₃H, —(CH₂)ₐOSO₃⁻l, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃⁼, —(CH₂)ₐOPO₃H₂, —(CH₂)ₐOPO₃H⁻ and —(CH₂)ₐOPO₃ where a is an integer from 1 to 10. In one example of this embodiment, the EDG(s) are independently C₁-C₁₀ alkyl, —NR⁴⁸R⁴⁹, —OR⁴⁶, —NR⁵⁰COR⁵¹, or —SR⁴⁷, wherein R⁴⁶-R⁵¹ are as described in the context of compounds of formula (FX1). In an embodiment, an EDG is located at the terminus of a substituent arm of the dithienofuran core of the present compounds.

When used herein, the terms “diagnosis”, “diagnostic” and other root word derivatives are as understood in the art and are further intended to include a general monitoring, characterizing and/or identifying a state of health or disease. The term is meant to encompass the concept of prognosis. For example, the diagnosis of cancer can include an initial determination and/or one or more subsequent assessments regardless of the outcome of a previous finding. The term does not necessarily imply a defined level of certainty regarding the prediction of a particular status or outcome.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids.

As defined herein, “administering” means that a compound or formulation thereof of the present invention, such as an optical agent, is provided to a patient or subject, for example in a therapeutically effective amount. The present invention includes methods for a biomedical procedure wherein a therapeutically or diagnostically effective amount of a compound having any one of formulae (FX1)-(FX4) is administered to a patient in need of treatment, for example to a patient undergoing treatment for a diagnosed diseased state including cancer and vascular diseases. Administering may be carried out by a range of techniques known in the art including intravenous, intraperitoneal or subcutaneous injection or infusion, oral administration, transdermal absorption through the skin, or by inhalation.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group linked to oxygen and can be represented by the formula R—O. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cyclic alkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9- or 10-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogens replaced with one or more fluorine atoms.

Aryl groups include groups having one or more 5-, 6- or 7-member aromatic or heterocyclic aromatic rings. Aryl groups can contain one or more fused aromatic rings. Heterocyclic aromatic rings can include one or more N, O, or S atoms in the ring. Heterocyclic aromatic rings can include those with one, two or three N, those with one or two O, and those with one or two S, or combinations of one or two or three N, O or S. Aryl groups are optionally substituted. Substituted aryl groups include among others those which are substituted with alkyl or alkenyl groups, which groups in turn can be optionally substituted. Specific aryl groups include phenyl groups, biphenyl groups, pyridinyl groups, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following benzene, naphthalene, naphthoquinone, diphenylmethane, fluorene, anthracene, anthraquinone, phenanthrene, tetracene, naphthacenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic radical, including monovalent, di valent and polyvalent radicals, of the aromatic and heterocyclic aromatic groups listed above provided in a covalently bonded configuration in the compounds of the present invention. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl having one or more hydrogens replaced with one or more fluorine atoms, chlorine atoms, bromine atoms and/or iodine atoms.

Optional substitution of any alkyl, alkenyl and aryl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for alkyl, alkenyl and aryl groups include among others:
—COOR where R is a hydrogen or an alkyl group or an aryl group and more specifically where R is methyl, ethyl, propyl, butyl, or phenyl groups all of which are optionally substituted;
—COR where R is a hydrogen, or an alkyl group or an aryl groups and more specifically where R is methyl, ethyl, propyl, butyl, or phenyl groups all of which groups are optionally substituted;
—CON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;
—OCON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;
—N(R)₂ where each R, independently of each other R, is a hydrogen, or an alkyl group, acyl group or an aryl group and more specifically where R is methyl, ethyl, propyl, butyl, or phenyl or acetyl groups all of which are optionally substituted; or R and R can form a ring which may contain one or more double bonds.
—SR, —SO₂R,or —SOR where R is an alkyl group or an aryl groups and more specifically where R is methyl, ethyl, propyl, butyl, phenyl groups all of which are optionally substituted; for —SR, R can be hydrogen;
—OCOOR where R is an alkyl group or an aryl groups;
—SO₂N(R)₂ where R is a hydrogen, an alkyl group, or an aryl group and R and R can form a ring;
—OR where R is H, alkyl, aryl, or acyl; for example, R can be an acyl yielding —OCOR* where R* is a hydrogen or an alkyl group or an aryl group and more specifically where R* is methyl, ethyl, propyl, butyl, or phenyl groups all of which groups are optionally substituted.

As used herein, the term “alkylene” refers to a divalent radical derived from an alkyl group as defined herein. Alkylene groups in some embodiments function as attaching and/or spacer groups in the present compositions. Compounds of the present invention include substituted and unsubstituted C₁-C₂₀ alkylene, C₁-C₁₀ alkylene and C₁-C₅ alkylene groups.

As used herein, the term “cycloalkylene” refers to a divalent radical derived from a cycloalkyl group as defined herein. Cycloalkylene groups in some embodiments function as attaching and/or spacer groups in the present compositions. Compounds of the present invention include substituted and unsubstituted C₁-C₂₀ cycloalkylene, cycloalkylene and C₁-C₅ cycloalkylene groups.

As used herein, the term “alkenylene” refers to a divalent radical derived from an alkenyl group as defined herein. Alkenylene groups in some embodiments function as attaching and/or spacer groups in the present compositions. Compounds of the present invention include substituted and unsubstituted C₁-C₂₀ alkenylene, C₁-C₁₀ alkenylene and C₁-C₅ alkenylene groups.

As used herein, the term “cylcoalkenylene” refers to a divalent radical derived from a cylcoalkenyl group as defined herein. Cycloalkenylene groups in some embodiments function as attaching and/or spacer groups in the present compositions. Compounds of the present invention include substituted and unsubstituted C₁-C₂₀ cylcoalkenylene, C₁-C₁₀ cylcoalkenylene and C₁-C₅ cylcoalkenylene groups.

As used herein, the term “alkynylene” refers to a divalent radical derived from an alkynyl group as defined herein. Alkynylene groups in some embodiments function as attaching and/or spacer groups in the present compositions. Compounds of the present invention include substituted and unsubstituted C₁-C₂₀ alkynylene, C₁-C₁₀ alkynylene and C₁-C₅ alkynylene groups.

As used herein, the term “halo” refers to a halogen group such as a fluoro (—F), chloro (—Cl), bromo (—Br) or iodo (—I).

As used herein, the term “azide” refers to a group having one or more —N₃ moieties. Azide groups useful in the present compounds include acyclic and cyclic aliphatic groups and aromatic groups having a —N₃ moiety provided as a substituent. In an embodiment, for example, an azide group of a compound of the present invention includes a C₅-C₂₀ aryl, optionally a C₅-C₁₀ aryl, having an —N₃ moiety provided as the terminus of a substituent arm of a carbocyclic or heterocyclic aromatic ring. In an embodiment, for example, an azide group of a compound of the present invention is a phenyl group, pyrazine group, azulene group or aza-azulene group having an —N₃ moiety provided as the terminus of a substituent arm of the aromatic ring or fused ring structure. In an embodiment, the invention provides a compound of any of formula (FX1)-(FX4) having —N₃ directly or indirectly linked via W¹-W⁴, and optionally L¹-L⁴, to the to the dithienofuran core of the compound.

As used herein, the term “azo” refers to a group having at least one —N═N— moiety. Azo groups useful in the present compounds include acyclic and cyclic groups having an —N═N— moiety, including: (i) aryl-azo groups having an —N═N— moiety directly or indirectly linked to one or more carbocyclic or heterocyclic aromatic rings of a C₅-C₂₀ aryl, (ii) alkyl-azo groups having an —N═N— moiety directly or indirectly linked to a C₁-C₂₀ alkyl group and (iii) alkylaryl-azo groups having an —N═N— moiety directly or indirectly linked to a C₁-C₂₀ alkyl group and one or more carbocyclic or heterocyclic aromatic rings of a C₅-C₂₀ aryl. In an embodiment, for example, an azo group of a compound of the invention includes an acyclic or cyclic aliphatic group, such as a C₁-C₂₀ alkyl or C₂-C₂₀ alkenyl group, optionally a C₁-C₁₀ alkyl or C₂-C₁₀ alkenyl group, wherein at least one carbon-carbon bond or carbon-carbon double bond is replaced with a nitrogen-nitrogen double bond (i.e. —N═N—). In an embodiment, for example, an azo group of a compound of the invention includes an alicyclic group wherein a carbon-carbon bond in a aliphatic carbocyclic or heterocyclic ring is replaced with a nitrogen-nitrogen double bond (i.e. —N═N—). In an embodiment, for example, an azo group of a compound of the invention includes a fused ring structure comprising one or more aromatic groups and one or more aliphatic groups, wherein a carbon-carbon bond in a carbocyclic or heterocyclic ring of the aliphatic group is replaced with a nitrogen-nitrogen double bond (i.e. —N═N—).

As an example, the invention provides a compound of any of formula (FX1)-(FX4) having an azo group directly or indirectly linked via W¹-W⁴, and optionally L¹-L⁴, to the to the dithienofuran core of the compound, wherein the azo group has the formula (FX13), (FX14), (FX15) or (FX16):

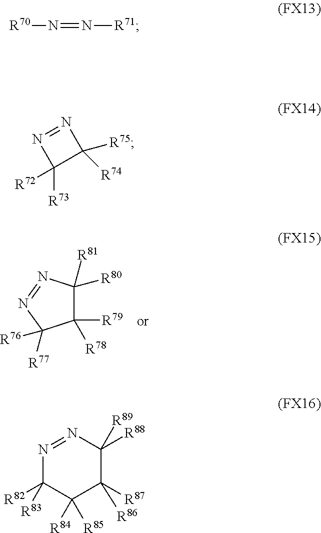

wherein at least one of R⁷⁰-R⁸⁹ connects the azo group directly or indirectly to the dithienofuran core of the compound; wherein each of the others of R⁷⁰-R⁸⁹ is independently hydrogen, C₁-C₂₀ alkyl, or C₅-C₂₀ aryl, or wherein or any two adjacent of the others of R⁷⁰-R⁸⁹ combine to form one or more carbocyclic or heterocyclic 4, 5, 6, or 7 membered alicyclic or aromatic rings.

As used herein, the term “diazo” refers to a group having one or more —C═N═N moieties. Diazo groups useful in the present compounds include acyclic and cyclic aliphatic groups and aromatic groups having a —C═N═N moiety provided as a substituent. In an embodiment, for example, a diazo group of a compound of the present invention includes a C₅-C₂₀ aryl, optionally a C₅-C₁₀ aryl, having an —C═N═N moiety provided as the terminus of a substituent arm of a carbocyclic or heterocyclic aromatic ring. In an embodiment, for example, an diazo group of a compound of the present invention is a phenyl group, pyrazine group, azulene group or aza-azulene group having an —C═N═N moiety provided as the terminus of a substituent arm of the aromatic ring or fused ring structure. In an embodiment, the invention provides a compound of any of formula (FX1)-(FX4) having —C═N═N directly or indirectly linked via W¹-W⁴, and optionally L¹-L⁴, to the to the dithienofuran core of the compound.

As used herein, the term “oxaza” refers to a group having at least one —(R)N—O— moiety. Oxaza groups useful in the present compounds include acyclic and cyclic groups having an —(R)N—O— moiety, including: (i) aryl-oxaza groups having a —(R)N—O— moiety directly or indirectly linked to one or more carbocyclic or heterocyclic aromatic rings of a C₅-C₂₀ aryl, (ii) alkyl-oxaza groups having a —(R)N—O— moiety directly or indirectly linked to a C₁-C₂₀ alkyl group and (iii) alkylaryl-oxaza groups having a —(R)N—O— moiety directly or indirectly linked to a C₁-C₂₀ alkyl group and one or more carbocyclic or heterocyclic aromatic rings of a C₅-C₂₀ aryl. In an embodiment, for example, an oxaza group of a compound of the invention includes an acyclic or cyclic aliphatic group, such as a C₁-C₂₀ alkyl or C₂-C₂₀ alkenyl group, optionally a C₁-C₁₀ alkyl or C₂-C₁₀ alkenyl group, wherein at least one carbon-carbon bond or carbon-carbon double bond is replaced with a nitrogen-oxygen single bond (i.e. —(R)N—O—). In an embodiment, for example, an oxaza group of a compound of the invention includes an alicyclic group wherein a carbon-carbon bond in an aliphatic carbocyclic or heterocyclic ring is replaced with a nitrogen-oxygen single bond (i.e. —(R)N—O—). In an embodiment, for example, an oxaza group of a compound of the invention includes a fused ring structure comprising one or more aromatic groups and one or more aliphatic groups, wherein a carbon-carbon bond in a carbocyclic or heterocyclic ring of the aliphatic group is replaced with a nitrogen-oxygen single bond (i.e. —(R)N—O—).

As an example, the invention provides a compound of any of formula (FX1)-(FX4) having an oxaza group directly or indirectly linked via W¹-W⁴, and optionally L¹-L⁴, to the dithienofuran core of the compound, wherein the oxaza group has the formula (FX17), (FX18), (FX19) or (FX20):

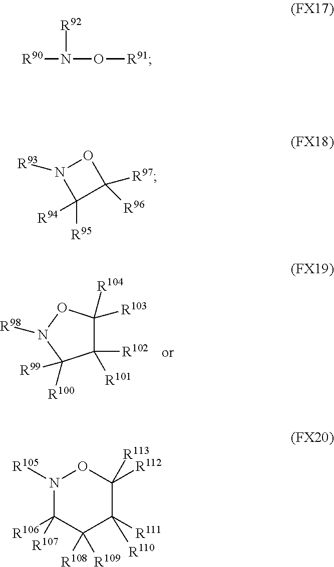

wherein at least one of R⁹-R¹¹³ connects the azo group directly or indirectly to the dithienofuran core of the compound; wherein each of the others of R⁹⁰-R¹¹³ is independently hydrogen, C₁-C₂₀ alkyl, or C₅-C₂₀ aryl, or wherein or any two adjacent of the others of R⁹⁰-R¹¹³ combine to form one or more carbocyclic or heterocyclic 4, 5, 6, or 7 membered alicyclic or aromatic rings.

As used herein, the term “diaza” refers to a group having at least one —(R)N—N(R)— moiety. Diaza groups useful in the present compounds include acyclic and cyclic groups having an —(R)N—N(R)— moiety, including: (i) aryl-diaza groups having an —(R)N—N(R)— moiety directly or indirectly linked to one or more carbocyclic or heterocyclic aromatic rings of a C₅-C₂₀ aryl, (ii) alkyl-diaza groups having an —(R)N—N(R)— moiety directly or indirectly linked to a C₁-C₂₀ alkyl group and (iii) alkylaryl-diaza groups having an —(R)N—N(R)— moiety directly or indirectly linked to a C₁-C₂₀ alkyl group and one or more carbocyclic or heterocyclic aromatic rings of a C₅-C₂₀ aryl. In an embodiment, for example, a diaza group of a compound of the invention includes an acyclic or cyclic aliphatic group, such as a C₁-C₂₀ alkyl or C₂-C₂₀ alkenyl group, optionally a C₁-C₁₀ alkyl or C₂-C₁₀ alkenyl group, wherein at least one carbon-carbon bond or carbon-carbon double bond is replaced with a nitrogen-nitrogen single bond (i.e. —(R)N—N(R)—). In an embodiment, for example, a diaza group of a compound of the invention includes an alicyclic group wherein a carbon-carbon bond in an aliphatic carbocyclic or heterocyclic ring is replaced with a nitrogen-nitrogen single bond (i.e. —(R)N—N(R)—). In an embodiment, for example, a diaza group of a compound of the invention includes a fused ring structure comprising one or more aromatic groups and one or more aliphatic groups, wherein a carbon-carbon bond in a carbocyclic or heterocyclic ring of the aliphatic group is replaced with a nitrogen-nitrogen single bond (i.e. —(R)N—N(R)—).

As an example, the invention provides a compound of any of formula (FX1)-(FX4) having a diaza group directly or indirectly linked via W¹-W⁴, and optionally L¹-L⁴, to the dithienofuran core of the compound, wherein the diaza group has the formula (FX21), (FX22), (FX23) or (FX24):

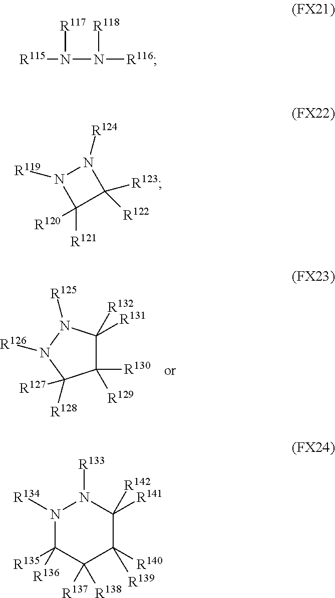

wherein at least one of R¹¹⁵-R¹⁴² connects the azo group directly or indirectly to the dithienofuran core of the compound; wherein each of the others of R¹¹⁵-R¹⁴² is independently hydrogen, C₁-C₂₀ alkyl, or C₅-C₂₀ aryl, or wherein or any two adjacent of the others of R¹¹⁵-R¹⁴² combine to form one or more carbocyclic or heterocyclic 4, 5, 6, or 7 membered alicyclic or aromatic rings.

As is customary and well known in the art, hydrogen atoms in formulae (FX1)-(FX4) are not always explicitly shown, for example, hydrogen atoms bonded to the carbon atoms of aromatic and alicyclic rings are not always explicitly shown in formulae (FX1)-(FX4).

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible. In addition, the compounds of this invention include all stereochemical isomers arising from the substitution of these compounds.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, -32-cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids, including but not limited to cysteine. Other pharmaceutically acceptable salts may be found, for example, in Stahl et al., Handbook of Pharmaceutical Salts: Properties, Selection, and Use, Wiley-VCH; Verlag Helvetica Chimica Acta, Zürich, 2002. (ISBN 3-906390-26-8). Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄³⁰ , where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

The compounds of this invention may contain one or more chiral centers. Accordingly, this invention is intended to include racemic mixtures, diasteromers, enantiomers and mixtures enriched in one or more steroisomer. The scope of the invention as described and claimed encompasses the racemic forms of the compounds as well as the individual enantiomers and non-racemic mixtures thereof.

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

In certain embodiments, the invention encompasses administering optical agents useful in the invention to a patient or subject. A “patient” or “subject”, used equivalently herein, refers to an animal. In particular, an animal refers to a mammal, preferably a human. The subject may either: (1) have a condition diagnosable, preventable and/or treatable by administration of an optical agent of the invention; or (2) is susceptible to a condition that is diagnosable, preventable and/or treatable by administering an optical agent of this invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

Compositions of the invention includes formulations and preparations comprising one or more of the present optical agents provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

In an embodiment, the invention provides a pharmaceutical formulation having an active ingredient comprising a composition of the invention, such as a compound of any one of formulae (FX1)-(FX4). In an embodiment, the invention provides a method of synthesizing a composition of the invention or a pharmaceutical formulation thereof, such as a compound of any one of formulae (FX1)-(FX4). In an embodiment, a pharmaceutical formulation comprises one or more excipients, carriers, diluents, and/or other components as would be understood in the art. Preferably, the components meet the standards of the National Formulary (“NF”), United States Pharmacopoeia (“USP”; United States Pharmacopeial Convention Inc., Rockville, Md.), or Handbook of Pharmaceutical Manufacturing Formulations (Sarfaraz K. Niazi, all volumes, ISBN: 9780849317521, ISBN 10: 0849317525; CRC Press, 2004). See, e.g., United States Pharmacopeia and National Formulary (USP 30-NF 25), Rockville, Md.: United States Pharmacopeial Convention; 2007; and 2008, and each of any earlier editions; The Handbook of Pharmaceutical Excipients, published jointly by the American Pharmacists Association and the Pharmaceutical Press (Pharmaceutical Press (2005) (ISBN-10: 0853696187, ISBN-13: 978-0853696186); Merck Index, Merck & Co., Rahway, N.J.; and Gilman et al., (eds) (1996); Goodman and Gilman's: The Pharmacological Bases of Therapeutics, 8th Ed., Pergamon Press. In embodiments, the formulation base of the formulations of the invention comprises physiologically acceptable excipients, namely, at least one binder and optionally other physiologically acceptable excipients. Physiologically acceptable excipients are those known to be usable in the pharmaceutical technology sectors and adjacent areas, particularly, those listed in relevant pharmacopeias (e.g. DAB, Ph. Eur., BP, NF, USP), as well as other excipients whose properties do not impair a physiological use.

In an embodiment, an effective amount of a composition of the invention is a therapeutically effective amount. As used herein, the phrase “therapeutically effective” qualifies the amount of compound administered in the therapy. This amount achieves the goal of ameliorating, suppressing, eradicating, preventing, reducing the risk of, or delaying the onset of a targeted condition. In an embodiment, an effective amount of a composition of the invention is a diagnostically effective amount. As used herein, the phrase “diagnostically effective” qualifies the amount of compound administered in diagnosis, for example of a disease state or other pathological condition. The amount achieves the goal of being detectable while avoiding adverse side effects found with higher doses. In an embodiment, an active ingredient or other component is included in a therapeutically acceptable amount. In an embodiment, an active ingredient or other component is included in a diagnostically acceptable amount.

Variations on compositions including salts and ester forms of compounds: Compounds of this invention and compounds useful in the methods of this invention include those of the compounds and formula(s) described herein and pharmaceutically-acceptable salts and esters of those compounds. In embodiments, salts include any salts derived from the acids of the formulas herein which acceptable for use in human or veterinary applications. In embodiments, the term esters refers to hydrolyzable esters of compounds of the names and structural formulas herein. In embodiments, salts and esters of the compounds of the formulas herein can include those which have the same or better therapeutic, diagnostic, or pharmaceutical (human or veterinary) general properties as the compounds of the formulas herein. In an embodiment, a composition of the invention is a compound or salt or ester thereof suitable for pharmaceutical formulations.

In an embodiment, the invention provides a method for treating or diagnosing a medical condition comprising administering to a subject (e.g. patient) in need thereof, a therapeutically effective amount or diagnostically effective amount of a composition of the invention, such as a compound of any one of formulae (FX1)-(FX4). In an embodiment, the medical condition is cancer, or various other diseases, injuries, and disorders, including cardiovascular disorders such as atherosclerosis and vascular restenosis, inflammatory diseases, ophthalmic diseases and dermatological diseases.

In an embodiment, the invention provides a medicament which comprises a therapeutically effective amount of one or more compositions of the invention, such as a compound of any one of formulae (FX1)-(FX4). In an embodiment, the invention provides a medicament which comprises a diagnostically effective amount of one or more compositions of the invention. In an embodiment, the invention provides a method for making a medicament for treatment of a condition described herein. In an embodiment, the invention provides a method for making a medicament for diagnosis or aiding in the diagnosis of a condition described herein. In an embodiment, the invention provides the use of one or more compositions set forth herein for the making of a medicament.

Compounds of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds set forth herein.

In an embodiment, a composition of the invention is isolated or purified. In an embodiment, an isolated or purified compound may be at least partially isolated or purified as would be understood in the art.

The invention is further detailed in the following Examples, which are offered by way of illustration and are not intended to limit the scope of the invention in any manner.

### EXAMPLE 1

### Dithienofuran Dyes for Photodiagnostic Agents and Phototherapeutic Agents

### 1. a Composition Classes of Dithienofuran Dyes Photodiagnostic and Phototherapeutic Agents

Optical agents of the present invention include dyes, and derivatives thereof, having a fused ring dithienofuran core structure which is optionally derivatized to provide useful optical, biological, chemical and physical properties. Dithienofuran dyes of the present invention provide functionality as exogenous optical agents for biomedical and bioanalytical applications including imaging, visualization, diagnostic monitoring and phototherapeutic applications.

Optical agents of the present invention are optionally multifunctional agents capable of providing a useful combination of photodiagnostic, phototherapeutic, molecular recognition and/or targeting functionality. In an embodiment, for example, a dithienofuran dye component of the present compositions imparts useful optical functionality for optical agents of the present invention, for example by functioning as an optical absorber, chromophor, fluorophor, or energy transfer moiety. Optionally, optical agents of the present invention further comprise photosensitizer and/or targeting components. In an embodiment, for example, an optical agent of the present invention comprises a photosensitizer component integrated with a dithienofuran dye component to access enhanced administration, delivery and photoactivation functionality for phototherapy. Further, optical agents and bioconjugates thereof are provided having one or more targeting ligands covalently bonded to or noncovalently associated with a dithienofuran dye of the present invention, thereby providing specificity for administering, targeting, delivery and/or localizing an optical agent to a specific biological environment, such as a specific organ, tissue, cell type or tumor site.

Selection of R¹, R², R³, and R⁴ in the optical agents of formulae (FX1)-(FX4) establishes, at least in part, the physical, chemical, optical and/or pharmacokinetic properties of optical agents for the present compositions and methods. In some embodiments, for example, selection of the composition of R¹, R², R³, and R⁴ may be based, at least in part, on a number of pharmacokinetic and physical properties supporting effective delivery and clearance of the optical agents of the present methods and compositions. Such factors may include solubility, toxicity, immune response, biocompatibility, and bioclearance considerations. In some embodiments, any one of R¹, R², R³, and R⁴ comprises a hydrophilic group, a lipophilic group, hydrophobic group, or an amphiphilic group. In an embodiment, at least one of R¹, R², R³, and R⁴ is a substituent comprising poly(ethylene glycol) (PEG; —(CH₂CH₂O)_{b}—), or a derivative of PEG. In an embodiment, for example, the invention provides a composition having any of the formula (FX1)-(FX4), wherein at least one of R¹, R², R³, and R⁴ is a substituent comprising —(CH₂CH₂O)_{b}—, wherein b is selected from the range of 1 to 100. Optionally, compositions of the present invention comprise a plurality of poly(ethylene glycol) components, for example wherein more than one of R¹, R², R³, and R⁴ is a substituent comprising —(CH₂CH₂O)ₘ—, wherein m is selected from the range of 1 to 100. Incorporation of a poly(ethylene glycol) component in some compositions of the present invention provides pharmacokinetic, chemical, and/or physical properties useful for bioanalytical, diagnostic and/or phototherapeutic applications. Poly(ethylene glycol) containing compounds of some embodiments of the present invention, for example, provided enhanced biocompatibility, low toxicity and suppress immune responses upon administration. Poly(ethylene glycol) containing compounds of some embodiments of the present invention facilitate formulation, administration and/or delivery, for example, by enhancing solubility.

In some embodiments, for example, R¹, R², R³, and R⁴ are selected to provide optical properties supporting and enabling use of these compositions in imaging, photodiagnostic and phototherapeutic methods, such as providing one or more of the following: (i) strong absorption in the visible and/or infrared regions of the electromagnetic spectrum (e.g., 350 to 1300 nanometers, preferably for some applications 400-900 nanometers); (ii) a large Stokes's shift (e.g., 50-200 nanometers); (iii) a large fluorescence quantum yield (e.g., φ≧0.5); (iv) a large quantum yield for the production of reactive intermediates, such as radicals, ions, nitrene, carbine and singlet oxygen (¹O₂), capable of causing photoactivation initiated tissue damage. Selection of combinations of R¹, R², R³, and R⁴ providing electron donating group and electron withdrawing group pairs on the fused ring backbone of compounds of (FX1)-(FX4) is particularly useful for tuning the absorption and emission properties of optical agents in the present methods and compositions. In an embodiment, a dithienofuran dye having formula (FX1)-(FX4) is derivatized by the addition of at least one electron withdrawing group and at least one electron donating group bonded directly or indirectly to a carbon atom of the fused ring backbone. In an embodiment, for example, one or more of the electron withdrawing (EWG) and electron donating (EDG) group(s) are directly attached to the fused ring backbone. In another embodiment, EWG and EDG are indirectly attached to the ring through an unsaturated spacer or attaching moiety providing conjugation with the double bonds in the backbone. Electron donating and withdrawing groups in these dye compositions may be positioned ortho, meta or para to each other with respect to there relative position on the fused ring backbone. In some embodiments, for example, two electron withdrawing groups are positioned pare to each other on the fused ring backbone and two electron donating groups are positioned pare to each other on the fused ring backbone. In some embodiments, electron withdrawing groups and electron donating groups are positioned so as to increase the symmetry of the overall compound.

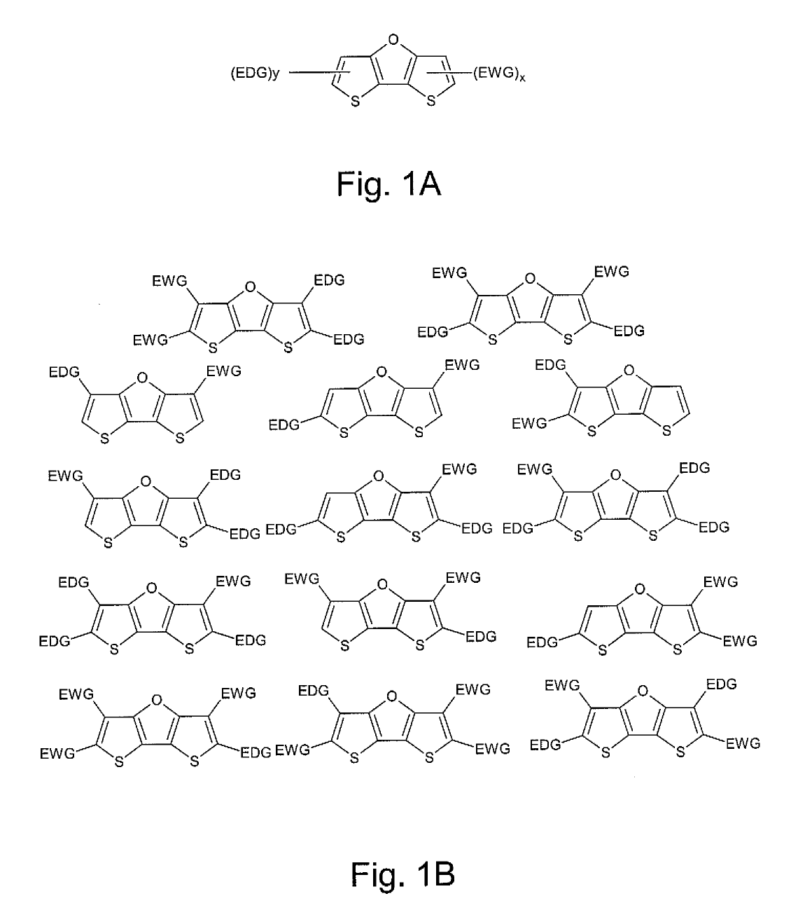

Derivatives of the present dithienofuran dyes having electron withdrawing group and electron donating group combinations, for example, are useful for providing dyes having excitation and emission properties useful for biomedical applications, such as excitation and emission spectra in the visible or NIR regions of the electromagnetic spectrum. In an embodiment, for example, one or more electron withdrawing and electron donating group(s) are bonded to the fused ring backbone through a resonance bond conjugating a chemically unsaturated linking moiety and the electron withdrawing and electron donating groups. Such “push-pull” optical agents of the present invention provide a conjugated bridge end-capped by electron-donor and electron-withdrawing groups which can provide enhanced absorption and quantum yield for fluorescence. The composition and position of substituents on the fused ring backbone of the present compounds may also be selected to provide “push pull” optical agents having excitation and emission spectra in the visible and NIR regions of the spectrum. FIG. 1A provides a chemical formula for a class of dithienofuran dyes of the present invention having a combination of electron withdrawing group(s) and electron donating group(s) bonded directly or indirectly to the fused ring backbone. FIG. 1B provides chemical formulae showing examples of specific arrangements and positions of electron withdrawing and electron donating groups useful in certain applications of the present invention. In FIG. 1A, EWG refers to an electron withdrawing group, EDG refers to an electron donating group, and x and y independently have values of 1 or 2. In FIG. 1A, y equal to 1 indicates a single EDG directly or indirectly bonded to the dithienofuran backbone and y equal to 2 indicates two EDGs directly or indirectly bonded to the dithienofuran backbone, for example bonded at two different carbons of the dithienofuran backbone. In FIG. 1A, x equal to 1 indicates a single EWG directly or indirectly bonded to the dithienofuran backbone and x equal to 2 indicates two EWGs directly or indirectly bonded to the dithienofuran backbone, for example bonded at two different carbons of the dithienofuran backbone. In the formulae provided in FIGS. 1A and 1B, the composition of each electron with drawing group (EWG) and each electron donating group (EDG) may be independently selected.

The optical agents of this example may contain additional functionalities that can be used to attach various types of biomolecules, synthetic polymers, and organized aggregates for targeted and/or selective delivery to various organs or tissues of interest. Examples of synthetic polymers include polyaminoacids, polyols, polyamines, polyacids, oligonucleotides, aborols, dendrimers, and aptamers. The present invention includes, but is not limited to, small dye biomolecule conjugates which provide advantages over nonspecific dyes or the conjugation of probes or photosensitive molecules to large biomolecules. These conjugates have enhanced localization and rapid visualization of tumors which is beneficial for both diagnosis and therapy. The agents are rapidly cleared from blood and non-target tissues so there is less concern for accumulation and for toxicity.

1 b. Synthesis of Dithienofuran Dyes for Photodiagnostic and Phototherapeutic Agents.

As will be apparent to those having skill in the art, compounds of the invention may be synthesized using a range of techniques and processes known in the art. For example, the synthesis of dithienofurans and dithienofuran derivatives is described and exemplified in: (1) Journal of Organic Chemistry 2008, 73(17) 6587-6594, and (ii) Bulletin of the Chemical Society of Japan 2004, 77(8), 1487-1497. Other references describing exemplary synthetic methods include: (i) Heterocyclic Chemistry, 4^{th} Ed., J. A. Joule and K. Mills, Blackwell Science Ltd., 2000, (ii) Heterocyclic Chemistry, Malcolm Sainsbury, The Royal Society of Chemistry, Thomas Graham House, Cambridge, 2001; and (iii) The Chemistry of Heterocycles: Structure, Reactions, Syntheses, and Applications, Theophil Eicher, Andreas Speicher, Siegfried Hauptmann, Wiley-VCH Gmbh & Co, Weinheim, 2003.

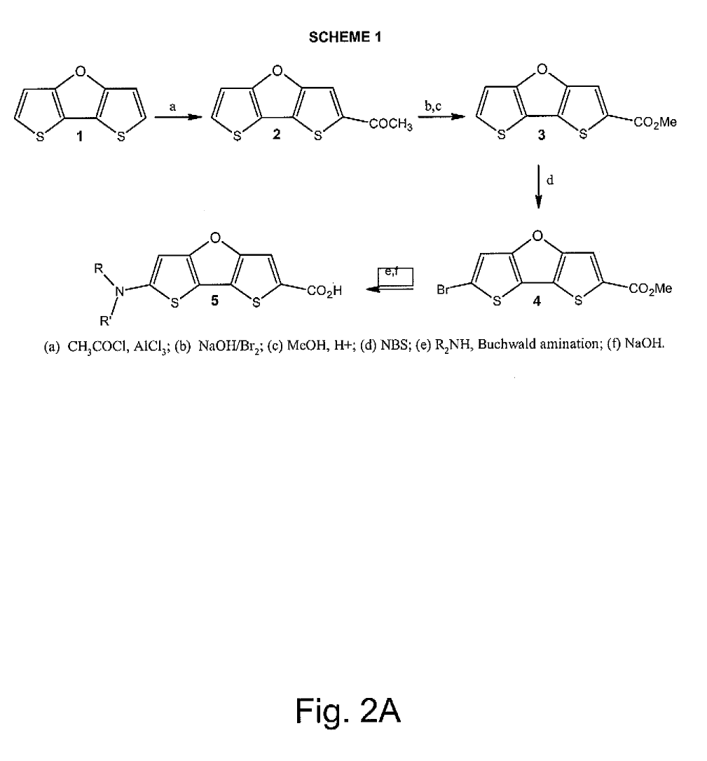

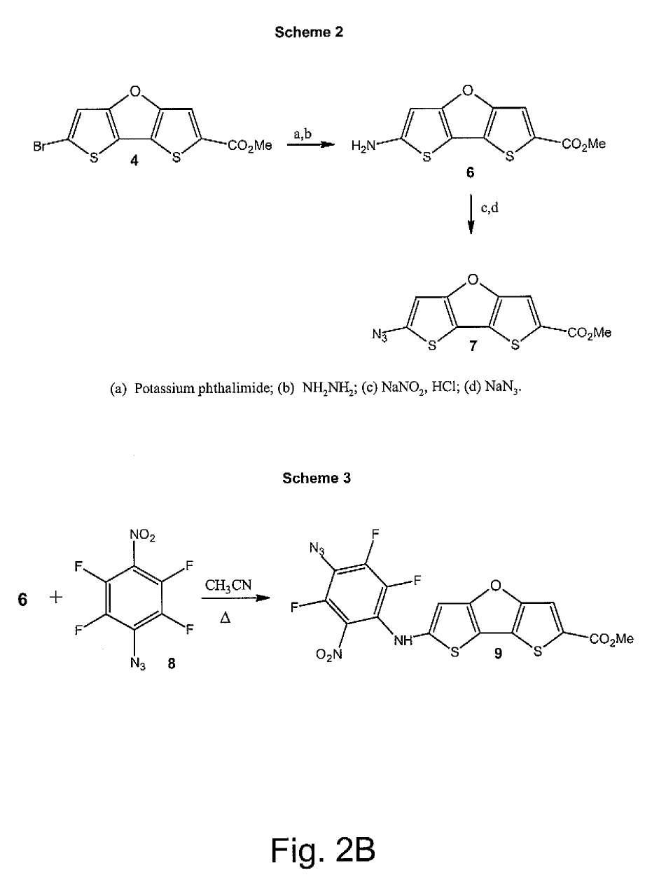

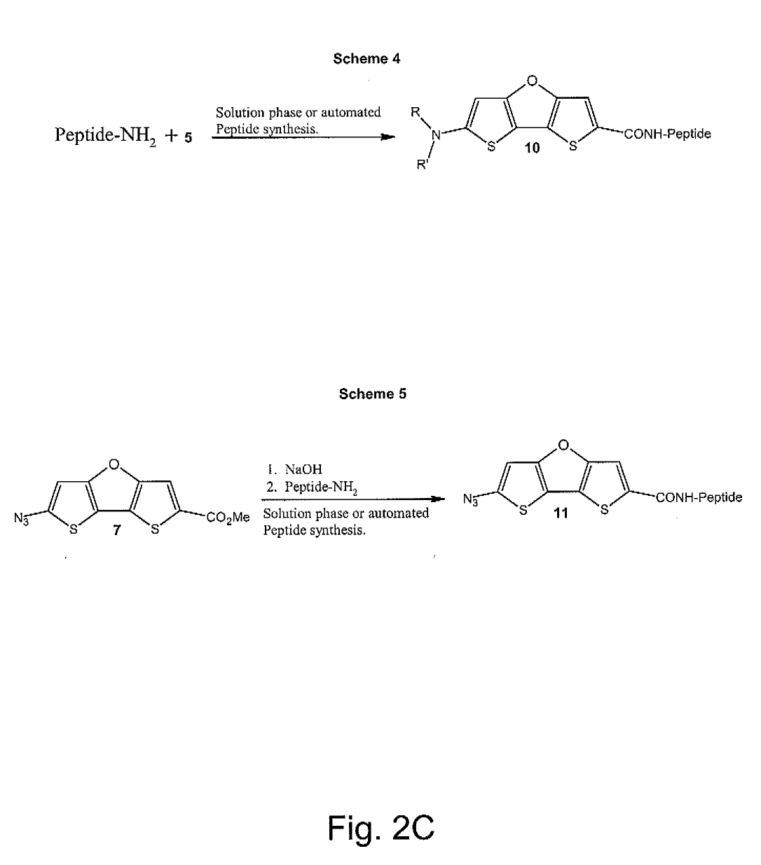

FIG. 2A provides Scheme 1, and corresponding experimental conditions, for synthesizing exemplary dithienofuran dyes of the present invention with “push-pull” electron donating and electron withdrawing groups.

FIG. 2B provides Scheme 2 and Scheme 3, and corresponding experimental conditions, for synthesizing exemplary dithienofuran compounds of the present invention having a photosensitizer component.

FIG. 2C provides Scheme 4 and Scheme 5, and corresponding experimental conditions, for synthesizing exemplary dithienofuran bioconjugates of the present invention having a ligand component for targeting.

### EXAMPLE 2

### Methods and Compositions for Imaging, Visualization, and Monitoring Physiological Function and Phototherapy

Optical agents of the present invention are highly versatile and provide a diagnostic platform useful for a variety of in vivo, in vitro and ex vivo diagnostic, visualization and imaging applications, such as, but not limited to, tomographic, photoacoustic and sonofluorescent imaging, monitoring and evaluating organ functioning, anatomical visualization, coronary angiography, and fluorescence endoscopy. A class of optical agents of the present invention, for example, is particularly useful for the detection, characterization and treatment of tumors and other lesions and/or abnormalities. In an embodiment, dithienofuran dyes of the present invention provide compositions for chemical and physiological sensing applications, for example, enabling the in situ, and real time monitoring of renal function in a patient. Some dithienofuran dyes of the present invention, for example, constitute optical probes, contrast agents and/or tracers for biomedical and bioanalytical applications. Optical agents of the present invention support a variety of therapeutic applications including phototherapeutic treatment methods, optical imaging and/or visualization guided surgery, administration and target specific delivery of therapeutic agents, and endoscopic procedures and therapies. In an embodiment, for example, dithienofuran dyes of the present compositions provide components for optical agents for absorbing electromagnetic radiation provided to a target biological environment, organ or tissue, and transferring it internally or externally to a phototherapeutic agent capable of achieving a desired therapeutic effect.

In the biomedical imaging, anatomical visualization, phototherapy and organ monitoring methods of the present invention, the agent may be introduced into the patient by any suitable method, including intravenous, intraperitoneal or subcutaneous injection or infusion, oral administration, transdermal absorption through the skin, or by inhalation. Some optical agents of the present invention provide detectable agents that can be administered to a subject and subsequently detected using a variety of optical techniques, including optical tomography, optical coherence tomography, fluorescence endoscopy, photoacoustic technology, sonofluorescence technology, light scattering technology, laser assisted guided surgery (LAGS), confocal microscopy, and one-, two-, three- and point optical detection.

2.a. Methods of Monitoring Organ Function Using Dithienofuran Compounds

The invention provides compositions and methods for monitoring organ function in a subject. In an embodiment, the present invention provides a method of using a detectable agent, the method comprising: (i) administering a diagnostically effective amount of a detectable agent to a subject, for example by administering the detectable agent into a bodily fluid of the subject, wherein the detectable agent is differentially separated from the bodily fluid by the organ or tissue; the detectable agent comprising a compound having formula (FX1):

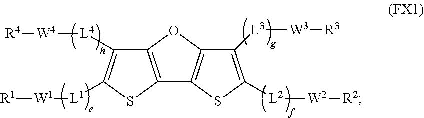

or a pharmaceutically acceptable salt or ester thereof, wherein: each of L¹, L², L³, and L⁴, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza 2,5-dioxocyclopentylene, 1,4-diazacyclohexylene, —(CH₂CH₂O)_{b}—, or —(CHOH)ₐ—; each of W¹, W², W³, and W⁴ is independently a single bond, —(CH₂)ₙ—, —(HCCH)ₙ—, —O—, —S—, —SO—, —SO₂—, —SO₃—, —OSO₂—, —NR¹¹—, —CO—, —COO—, —OCO—m —OCOO—, —CONR¹²—, —NR¹³CO—, —OCONR¹⁴—, —NR¹⁶COO—, —NR¹⁶CONR¹⁷—, —NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCOO(CH₂)ₙ—, —CONR₂₁(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, —NR²CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, or —CO(CH₂)ₙNR³³CO—; each of R¹, R², R³, and R⁴ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₆ alkoxycarbonyl, halo, halomethyl, dihalomethyl, trihalomethyl, —CO₂R⁴⁰, —SOR⁴¹, —OSR⁴², —SO₂OR⁴³, —CH₂(CH₂OCH₂)ₒCH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —CH(R⁶²)CO₂H, —CH(R⁶³)NH₂, —N₃, FL or Bm; each of a and b is independently an integer selected from the range of 1 to 100; each of n is independently an integer selected from the range of 1 to 10; each of e, f, g and h is independently 0 or 1; each of R¹¹-R³³ is independently hydrogen, C₁-C₂₀ alkyl, or C₆-C₂₀ aryl; each of R⁴⁰-R⁶¹ is independently hydrogen or C₁-C₁₀ alkyl; each of R⁶² and R⁶³ is independently a side chain residue of a natural α-amino acid; each of FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound; and each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising ‘I to 50 nucleic acid units, a polypeptide comprising 2 to 30 amino acid units, or an echogenic agent; (ii) exposing the detectable agent in the bodily fluid to electromagnetic radiation for exciting emission from the detectable agent; (iii) measuring the emission from the detectable agent that is in the bodily fluid; and (iv) determining the physiological function of the organ or tissue of the subject based on measurement of the emission. In an embodiment, for example, the organ or tissue is a kidney, or tissue or cells thereof, of the subject. in an embodiment, for example, the organ or tissue is a liver, or tissue or cells thereof, of the subject.

In an embodiment, the methods of monitoring organ function of the invention comprises administering to a patient a compound having any one of formula selected from (FX1)-(FX4), including any of the specific compositions classes and compounds described in connection with formula (FX1)-(FX4). As will be understood by one of skill in the art, the present methods of monitoring organ function expressly include methods of using optical agents wherein the detectable agent includes the compound classes, compounds, and all variations thereof, described herein, including the compound classes, compounds and variations described in connection with any one of formulae (FX1)-(FX4).

In an embodiment, for example, the method further comprises exciting and measuring fluorescence from the detectable agent in the subject for a plurality of times after administration of the detectable agent. In an embodiment, a temporal profile of fluorescence form the detectable agent administered to the subject is determined and evaluated with respect to characterizing organ functioning, for example, by measuring a rate of change in fluorescence (e.g., a decrease in fluorescence) as a function of time, and optionally comparing the measured rate of change in fluorescence to a rate of change characteristic of a subject having a healthy organ or a subject having a known disease condition. Organ function can be assessed in the present methods by comparing differences in the manner in which normal and impaired cells remove the detectable agent (also refer to as a tracer in this context) from the bloodstream, by measuring the clearance or accumulation of these tracers in the organs or tissues, and/or by obtaining tomographic images of the organs or tissues. Blood pool clearance may be measured non-invasively from convenient surface capillaries such as those found in an ear lobe or a finger or can be measured invasively using an endovascular catheter. Accumulation of the tracer within the cells of interest can be assessed in a similar fashion. The clearance of the tracer compounds can be determined by selecting excitation wavelengths and filters for the emitted photons. The concentration vs time curves and/or fluorescence intensity vs time curves may be analyzed (preferably, but not necessarily in real time) by a microprocessor or the like.

Systems and methods of the present invention may optionally include an optical monitoring assembly or device for detecting optical agents of the invention. An example of an in vivo disease state optical monitoring assembly includes a source of electromagnetic radiation, an electromagnetic radiation detector and a data processing system. The electromagnetic radiation source generally includes or is interconnected with an appropriate device or devices for exposing at least a portion of a patient's body to electromagnetic radiation there from. Examples of appropriate devices that may be operatively connected to, or be a part of, the electromagnetic radiation source include, but are not limited to, catheters, endoscopes, fiber optics, ear clips, hand bands, head bands, forehead sensors, surface coils, and finger probes. indeed, any of a number of devices capable of emitting visible and/or near infrared electromagnetic radiation may be employed in a optical monitoring assembly.

The electromagnetic radiation detector of the optical monitoring assembly may be any appropriate system capable of collecting, detecting and measuring the intensity of electromagnetic radiation emitted from a subject. The electromagnetic radiation detector may be operatively connected to, for example, one or more optical collection elements. The optical collection elements of the optical monitoring assembly may include, among other elements, lenses, mirrors, optical filters (e.g., band pass filters and cut off filters), and fiber optics. Electromagnetic radiation detectors suitable for use with the disease state optical monitoring assembly include, but are not limited to, CCD detectors, CMOS detectors, photodiode detectors, photodiode array detectors, and photomultiplier tube detectors.

The data processing system of the optical monitoring assembly may be any appropriate system capable of processing data obtained from the electromagnetic radiation detector. For instance, the data processing system may include an amplifier (e.g., to amplify an electrical signal from the detector), and a processing unit (e.g., to process the electrical signal from the detector). The data processing system is preferably configured to manipulate collected electromagnetic radiation data and generate an intensity as a function of time profile and/or a concentration as a function of time curve indicative of clearance of an optical agent, conjugate, bioconjugate or integrated bioconjugate composition of the present invention from a subject. Indeed, the data processing system may be configured to generate appropriate disease state or health state data by comparing differences in amount of normal and impaired cells in the bloodstream, to determine a rate or an accumulation of the composition in cells, organs or tissues of the subject, and/or to provide tomographic images of cells, organs or tissues having the optical agent, conjugate, bioconjugate or integrated bioconjugate composition associated therewith.

In one protocol for optical monitoring, an effective amount of a composition having formula (FX1)-(FX4) including an optical agent, conjugate, bioconjugate or integrated bioconjugate of the invention is administered to the subject. At least a portion of the body of the subject is exposed to visible and/or near infrared electromagnetic radiation from the electromagnetic radiation source. For instance, the electromagnetic radiation from the electromagnetic radiation source may be delivered via a fiber optic that is affixed to an ear of the subject. The subject may be exposed to electromagnetic radiation from the electromagnetic radiation source before, during or after administration of the composition to the subject. In some cases, it may be beneficial to generate a background or baseline reading of electromagnetic radiation being emitted from the body of the subject, due to exposure to the electromagnetic radiation from the electromagnetic radiation source, before administering the composition to the subject. When the optical agents, conjugates, bioconjugates or integrated bioconjugates of the composition that are in the body of the subject are exposed to the electromagnetic radiation from the electromagnetic radiation source, the optical agents, conjugates, bioconjugates or integrated bioconjugates emit electromagnetic radiation that is collected by optical collection elements and detected by the electromagnetic radiation detector. The signal from the electromagnetic radiation detector is then analyzed by the data processing system.

Initially, administration of the composition to the subject generally enables an electromagnetic radiation signal indicative of the content of the optical agent(s), conjugate(s), bioconjugate(s) or integrated bioconjugate(s) in the subject. In some embodiments, the electromagnetic radiation signal tends to decay as a function of time as the optical agent(s), conjugate(s), bioconjugate(s) or integrated bioconjugate(s) is cleared from the subject. In a subject with a healthy disease state, the electromagnetic radiation signal will decay to near the baseline level as the optical agent(s), conjugate(s), bioconjugate(s) or integrated bioconjugate(s) is cleared from the subject. In a subject with an unhealthy disease condition, the optical agent(s), conjugate(s), bioconjugate(s) or integrated bioconjugate(s) will attach to cells, tissues or organs affected with a disease condition and will not be cleared by the subject during the time scale of the monitoring, or will be cleared at a rate which differs from the healthy disease state clearance rate. As a result, the electromagnetic radiation signal may decay at a different rate. Alternatively, the electromagnetic radiation signal may not decrease to the baseline level, but will remain at an elevated level. The difference between this increased electromagnetic radiation signal level (or decay rate) and the baseline level (or decay rate) may be indicative of a disease state in the subject. Some methods of the present invention further comprise comparing the rate of decay of fluorescence intensity at a number of different times so as to assess the state of organ function. As such, the subject may be exposed to the electromagnetic radiation from the electromagnetic radiation source for any amount of time appropriate for providing the desired disease state monitoring data. Likewise, the electromagnetic radiation collection, detection, and data processing systems may be allowed to collect and detect electromagnetic radiation for any amount of time appropriate for providing the desired disease state monitoring data.

In addition to noninvasive techniques, a modified pulmonary artery catheter that can be used to make desired measurements has been developed. This is a distinct improvement over current pulmonary artery catheters that measure only intravascular pressures, cardiac output and other derived measures of blood flow. Current critically ill patients are managed using these parameters but rely on intermittent blood sampling and testing for assessment of renal function. These laboratory parameters represent discontinuous data and are frequently misleading in many patient populations. Yet, importantly, they are relied upon heavily for patient assessment, treatment decisions, and drug dosing.

The modified pulmonary artery catheter incorporates an optical sensor into the tip of a standard pulmonary artery catheter. This wavelength-specific optical sensor can monitor the renal function specific elimination of a designed optically detectable chemical entity. Thus, by a method substantially analogous to a dye dilution curve, real-time renal function can be monitored by the disappearance of the optically detected compound. Appropriate modification of a standard pulmonary artery catheter generally includes merely making the fiber optic sensor wavelength-specific. Catheters that incorporate fiber optic technology for measuring mixed venous oxygen saturation exist currently.

In an embodiment of this aspect, the present invention provides a method of monitoring a physiological state or condition of a patient undergoing treatment. In this method, an effective amount of an optical agent of the present invention is administered to a mammal (e.g., a patient undergoing treatment). Further, the optical agent that has been administered is exposed to electromagnetic radiation. In addition, electromagnetic radiation transmitted, scattered or emitted by the optical agent is detected. In some embodiments, a change in the wavelengths or intensities of electromagnetic radiation emitted by the optical agent that has been administered to the mammal may be detected and/or measured, optionally as a function of time. Methods of this aspect of the present invention include in situ, real time methods of monitoring renal function in the mammal, wherein the optical agent is cleared by the renal system of the subject. Methods of this aspect of the present invention include in situ, real time methods of monitoring hepatic function in the mammal, wherein the optical agent is cleared by the hepatic system of the subject.

In an embodiment particularly useful for monitoring physiological function of an organ or tissue of a subject, the method of this aspect further comprises: (i) exposing the detectable agent in the bodily fluid to electromagnetic radiation for exciting emission from the detectable agent; (ii) measuring the emission from the detectable agent that is in the bodily fluid; and (iii) determining the physiological function of the organ or tissue of the subject based on measurement of the emission. The present invention includes fluorescence detection of an agent which is cleared from the bloodstream by the kidneys or liver. Thus, assessment of renal or hepatic function by in vivo fluorescence detection is encompassed within the scope of the invention. The invention can also be used to monitor the efficiency of hemodialysis. The organ or tissue in some methods is a kidney, or tissue or cells thereof, of the subject, wherein the present invention provides methods for monitoring renal function of the subject. The organ or tissue in some embodiments is a liver, or tissue or cells thereof, of the subject, wherein the present invention provides methods for monitoring hepatic function of the subject.

Methods of this aspect of the present invention may further comprise a variety of optional steps, including analysis of the measured emission from the optical agent as a function of time, such as over a period ranging from 10 minutes to 48 hours. In an embodiment, for example, the method further comprises measuring a blood clearance parameter or profile of the detectable agent administered to the subject. A method of this aspect further comprises comparing the blood clearance parameter or profile of the detectable agent administered to the subject to a reference blood clearance parameter or profile. Useful blood clearance parameters for this aspect of the invention include instantaneous and/or average rates of clearance of the detectable agent A method of this aspect further comprises comparing the emission from the subject or function thereof with one or more emission reference values or a function thereof of a reference subject. In some embodiments, measuring the emission from the detectable agent comprises measuring emission from the detectable agent in the bodily fluid at a plurality of different times. The clearance of a plurality of separate tracers may be determined simultaneously by selecting excitation wavelengths and filters for the emitted electromagnetic radiation. The concentration vs time or fluorescence intensity vs time curves may be analyzed in real time by a microprocessor. The resulting clearance rates may be calculated and displayed for immediate clinical impact. In cases where unlabeled competing compounds are present (e.g., LDL, asialoglycoproteins), a single blood sample may be analyzed for the concentration of these competing compounds and the results used to calculate a flux (micromoles/minute) through the clearance pathways.

In accordance with one embodiment of the present invention, a method is disclosed for determining cell and/or organ function by measuring the blood pool clearance of a targeted optical agent, sometimes referred to herein as a tracer. The cell and/or organ function can be determined by the rate these cells remove the tracer from the bloodstream. Function can also be assessed by measuring the rate the cells of interest accumulate the tracer or convert it into an active or other form. The agent may be targeted to a group of cells or organ which is a high capacity clearance system. The agent may be an optical agent comprising a dithienofuran dye, or derivative or conjugate thereof including bioconjugate, such as the compositions provided in formulae (FX1)-(FX4). For optical agents containing a dithienofuran dye component, blood pool clearance may be measured using a light source-photodetector device that measures tissue absorbance or fluorescence in a non-target site, such as an ear lobe, finger, brain or retina. Accumulation of the tracer within the cells of interest can be assessed in a similar fashion. The detection of such accumulation can be facilitated by using fluorophores which emit in the near infrared wavelengths since body tissues are relatively transparent at these wavelengths.

The present invention may be used for rapid bedside evaluation of biologic functions. For example, data on cardiac output, cause of hypercholesterolemia, as well as renal and hepatic function, may be obtained in less than sixty minutes at the bedside after a single intravenous injection. In accordance with one embodiment, a patient may receive a bolus injection of a plurality (e.g., three) of different compounds, each containing a different optical agent (e.g., fluorophore, dye, chromophore).

In an embodiment, the method comprises exposing the detectable agent in the bodily fluid to electromagnetic radiation having wavelengths selected over the range of 350 nm to 1300 nm. Optionally, excitation is achieved using electromagnetic radiation substantially free (e.g., less than about 10% of total radiant energy), of ultraviolet radiation for example to minimize exposure of the subject to electromagnetic radiation capable of causing unwanted cell or tissue damage. Excitation of optical agents may be provided by a wide range of techniques and optical sources as known in the art, including use of laser, fiber optic and/or endoscopic optical sources and methods. The present invention includes methods using multiphoton excitation of optical agents. In an embodiment, the method comprises measuring fluorescence from the detectable agent having wavelengths selected over the range of 350 nm to 1300 nm. Detection of emission, including fluorescence, can be achieved by wide a range of techniques and detection systems as known in the art, including detection by eye (e.g., visualization) and two-dimensional or three-dimensional detection.

2b. Methods for Phototherapy Using Dithienofuran Compounds

Phototherapy, such as photodynamic therapy (PDT), typically employs a combination of a nontoxic photosensitizer (PS) and visible or near infrared light to generate reactive species that kill or otherwise degrade target cells, such as tumors or other lesions. The present invention provides phototherapeutic agents useful for phototherapy.

The invention includes phototherapy methods wherein a phototherapeutic agent comprising a compound of any one of the formulae (FX1)-(FX4) is administered to a patient, for example, wherein a therapeutically effective amount of such a component is administered to a patient in need of treatment. In some embodiments, compounds of the invention provide an optical agent capable of selective targeting and delivery to a target tissue such as a tumor, site of inflammation or other lesion. Upon administration, the phototherapeutic agent is optionally allowed to accumulate in a target region of interest (e.g., target tissue, tumor, or organ). To induce selective tissue damage, the phototherapeutic agent is activated by exposure to electromagnetic radiation. In an embodiment, the phototherapeutic agent is activated after an effective concentration of the phototherapeutic agent has accumulated in a target tissue. An effective concentration of a compound of the invention depends on the nature of the formulation, method of delivery, target tissue, activation method and toxicity to the surrounding normal non-target tissue. Exposure to electromagnetic radiation and activation of the phototherapeutic agent may occur during or after administration of the phototherapeutic agent and accumulation at the target tissue.

For photoactivation, the target region is illuminated with electromagnetic radiation having a wavelength in the range of about 350 nm to about 1300 nm, preferably for some applications in the range of about 350 nm to about 900 nm. In some embodiments, the wavelength of the electromagnetic radiation corresponds to a peak in the absorption spectrum of the phototherapeutic agent, for example is within 20 nanometers of a peak in the absorption spectrum of the phototherapeutic agent in the visible or NIR regions. In some phototherapeutic procedures the target site is exposed to electromagnetic radiation having sufficient fluence and/or power sufficient to activate the phototherapeutic agent so as to induce cell death, for example via necrosis or apoptosis processes. In some embodiments, electromagnetic radiation having low energy, power or fluence is provided to activate the phototherapeutic agent without undesirable thermal effects. If the region of interest is, for example, a lesion or tumor on the skin surface, the region can be directly illuminated. Otherwise, endoscopic and/or endoluminal catheters equipped with an electromagnetic radiation source may be employed to provide a photodiagnostic and/or phototherapeutic effect.

Appropriate power and intensity of the electromagnetic radiation depends on the size, depth, and the pathology of the lesion, as is known to one skilled in the art. In an embodiment, the fluence of the electromagentic radiation is preferably, but not always, kept below 200 mW/cm², optionally below 100 mW/cm², to minimize undesirable thermal effects. The intensity, power, and duration of the illumination, and the wavelength of the electromagnetic radiation may vary widely depending on the body location, the lesion site, the effect to be achieved, etc. In an embodiment, the power of the applied electromagnetic radiation is preferably is selected over the range of 1-500 mW/cm², optionally for some applications selected over the range of 1-200 mW/cm² and optionally for some applications selected over the range of 1-100 mW/cm². In an embodiment, the duration of the exposure to applied electromagentic radiation selected over the range of 1 second to 60 minutes, and optionally for some applications selected over the range of 1 second to 10 minutes.

In an embodiment, the invention provides a method of using a phototherapeutic agent, the method comprising: (i) administering a therapeutically effective amount of a phototherapeutic agent to a subject, the phototherapeutic agent comprising a compound being of the formula (FX1):

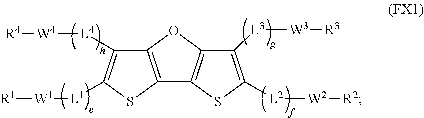

or a pharmaceutically acceptable salt or ester thereof, wherein: each of L¹, L², L³, and L⁴, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—; each of W¹, W², W³, and W⁴ is independently a single bond, —(CH₂)ₙ—, —(HCCH)ₙ—, —O—, —S—, —SO—, —SO₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, —NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCO(CH₂)ₙ—, OCOO(CH₂)ₙ—, —CONR²¹(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, —NR²⁶CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, -or —CO(CH₂)ₙNR³³CO—; each of R¹, R², R³, and R⁴ is independently a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₆ alkoxycarbonyl, halo, halomethyl, dihalomethyl, trihalomethyl, —CO₂R⁴⁰, —SOR⁴¹, —OSR⁴², —SO₂OR⁴³, —CH₂(CH₂OCH₂)_{c}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CN₂(CHOH)ₐR⁶⁰, —(CH₂CH₂O)_{b}R⁶¹, —CH(R⁶²)CO₂H, —CH(R⁶³)NH₂, —N₃, PS¹, PS², FL or Bm; wherein at least one of R¹-R⁴ is PS¹ or PS²; each of a and b is independently an integer selected from the range of 1 to 100; each of n is independently an integer selected from the range of 1 to 10; each of e, f, g and h is independently 0 or 1; each of R¹¹-R³³ is independently hydrogen, C₁-C₂₀ alkyl, or C₆-C₂₀ aryl; each of R⁴⁰-R⁶¹ is independently hydrogen or C₁-C₁₀ alkyl; each of R⁶² and R⁶³ is independently a side chain residue of a natural cc-amino acid; each of FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound; each PS¹ is independently a Type 1 photosensitizer; each PS² is independently a Type 2 photosensitizer; and each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent; and (ii) exposing the phototherapeutic agent administered to the patient to electromagnetic radiation. In an embodiment, the phototherapy methods of the invention comprise administering to a patient a compound having any one of formula selected from (FX1)-(FX4), including any of the specific compositions classes and compounds described in connection with formula (FX1)-(FX4), wherein at least one of R¹R⁴ is PS¹ or PS². In an embodiment, for example, the invention provide a method of using a phototherapeutic agent in a phototherapy procedure comprising administering to a subject a compound having any of formula (FX1)-(FX4), wherein at least one of R¹-R⁴ is PS¹, and optionally at least one of R¹-R⁴ is Bm. In an embodiment, for example, the invention provide a method of using a phototherapeutic agent in a phototherapy procedure comprising administering to a subject a compound having any of formula (FX1)-(FX4), wherein each PS¹ is an azide, azo, diazo, oxaza, or diaza group. In an embodiment, for example, the invention provide a method of using a phototherapeutic agent in a phototherapy procedure comprising administering to a subject a compound having any of formula (FX1)-(FX4), wherein at least one of R¹-R⁴ is PS², and optionally at least one of R¹-R⁴ is Bm. In an embodiment, for example, the invention provide a method of using a phototherapeutic agent in a phototherapy procedure comprising administering to a subject a compound having any of formula (FX1)-(FX4), wherein each PS² is a group corresponding to a porphyrin, benzoporphyrin, phthalocyanine, phenothiazine, chlorin, bacteriochlorin, phthalocyanine, porphyrin, purpurin, merocyanine, pheophorbides, psoralen, aminolevulinic acid (ALA), hematoporphyrin derivative, porphycene, porphacyanine, cyanine, indocyanine, phthalocyanine, rhodamine, phenoxazine, a phenoselenazine, fluorescein, squaraine, corrin, croconium, azo dye, methine dye, indolenium dye, halogen, anthracyline, C₁-C₂₀ peroxyalkyl, C₁-C₂₀ peroxyaryl, C₁-C₂₀ sulfenatoalkyl, sulfenatoaryl, naphthalocyanine, methylene blue, or chalcogenopyrylium analogue.

In an embodiment, the phototherapeutic agent is exposed to a therapeutically effective amount of electromagnetic radiation. As used herein, a therapeutically effective amount of electromagnetic radiation is an amount for achieving a desired therapeutic result, for example an amount for generating a therapeutically effective amount of reactive species for damaging or causing cell death of a selected target tissue. In an embodiment, the method further comprises generating one or more reactive species from said compound administered to the patient via the exposure of the phototherapeutic agent to applied electromagnetic radiation. In an embodiment, for example, the method further comprises the step of cleaving one or more photolabile bonds of the optical agent so as to generate reactive species comprising free radicals. In an embodiment, for example, the method further comprises the step of generating excited oxygen (e.g., singlet oxygen; ¹O₂) In an embodiment, the method further comprises targeting the phototherapeutic agent to a selected organ in the patient or to a selected tissue type in the patient. In an embodiment, a therapeutically effective dose of the phototherapeutic agent is administered to a patient in need of treatment.

Embodiments of this aspect may comprise a method of carrying out an in vivo therapeutic and/or diagnostic procedure. In an embodiment, the invention comprises a method of carrying out an in vivo phototherapeutic, photoactivation, and/or photosensitizing procedure. The present methods have broad clinical utility which includes, but is not limited to, phototherapy of tumors, inflammatory processes, and impaired vasculature. In embodiments, subjects of the invention may be any mammal, such as a human, and optionally the subject of the present methods is a patient in need of treatment and/or diagnosis. The present methods are also useful in ex vivo and in vitro procedures, including medical therapeutic and diagnostic procedures.

Methods of the invention may optionally further comprise a number of other steps. In an embodiment, the method further comprises the step of administering the phototherapeutic agent into a bodily fluid of the subject. The phototherapeutic agent may be introduced into the patient by any suitable method, including intravenous, intraperitoneal or subcutaneous injection or infusion, oral administration, transdermal absorption through the skin, or by inhalation. In an embodiment, the method further comprises contacting a target tissue, such as an organ, tissue, tumor, lesion, or cell type, with a compound of any one of formulae (FX1)-(FX4) prior to or during the exposure step. In an embodiment, the method further comprises allowing the compound to accumulate in a target tissue prior to exposure of the phototherapeutic agent to electromagnetic radiation. In an embodiment, the method further comprises contacting and/or selectively targeting the diagnostic agent to a selected organ, tissue, tumor, lesion, inflammation, or cell type. In an embodiment, the phototherapeutic agent is administered to the skin, a tumor, surgical site, or a wound site. In an embodiment, for example, the phototherapeutic agent is administered and/or delivered to a blood vessel, lung, heart, throat, ear, rectum, bladder, stomach, intestines, esophagus, liver, brain, prostrate, breast, or pancreas of the subject.

In an embodiment, dithienofuran dyes of the present invention provide carriers and antennae for Type I Phototherapeutic Agents. In an embodiment of this aspect, the dithienofuran dye is used as an “Antenna/Transducer” for absorbing the appropriate laser irradiation and transferring it internally (via FRET) to Type I phototherapeutic agents that are either physically associated with a dithienofuran dye or covalently attached to the dithienofuran dye. The type I phototherapeutic agent may be conjugatable derivatives of agents that decompose to cytotoxic reactive intermediates upon laser irradiation.

As will be understood by one having skill in the art, the optical conditions for the step of exposing the phototherapeutic agent administered to the patient to electromagnetic radiation will vary considerably with the (i) therapeutic and/or diagnostic objectives, and (ii) the condition of the subject (e.g., height, weight, state of health etc.). In an embodiment, the applied electromagnetic radiation has wavelengths, energy and/or fluence sufficient to achieve a desired therapeutic and/or diagnostic result. In an embodiment, the electromagnetic radiation has wavelengths, energy and/or fluence sufficient to activate the phototherapeutic agent, for example wavelengths, energy and/or fluence sufficient to result in generation of reactive species, including singlet oxygen and/or free radicals. In an embodiment, the electromagnetic radiation has wavelengths, energy and/or fluence sufficient to result in cleavage of at least one photolabile bond of the optical agent upon absorption. In an embodiment, the electromagnetic radiation exposed to the phototherapeutic agent has wavelengths corresponding to a maximum in the absorption spectrum of the phototherapeutic agent, preferably for some applications a maximum in the visible or NIR regions of the electromagnetic spectrum. Optionally, excitation is achieved using electromagnetic radiation substantially free (e.g., less than about 10% of total radiant energy), of ultraviolet radiation, for example, to minimize exposure of the subject to electromagnetic radiation capable of causing unwanted cell or tissue damage. Electromagnetic radiation may be provided to the phototherapeutic agent using a range of optical sources and/or surgical instrumentation, including a laser, light emitting diodes, fiber optic device, endoscope, catheter, optical filters, or any combination of these.

In an aspect, the optical agent comprises a dithienofuran dye of the present invention and a photosensitizer component, wherein exposure of the optical agent to electromagnetic radiation having a first wavelength distribution activates the phototherapeutic agent(s), thereby achieving a desired therapeutic effect, for example, by generating one or more reactive intermediates (e.g., free radicals, excited state oxygen (¹O₂), ions, nitrene, carbine etc.) capable of causing tissue damage. Optionally, the optical agent is first excited with electromagnetic radiation having a second wavelength distribution, that is different from the first distribution and is capable of exciting fluorescence from the dithienofuran dye component of the optical agent. This optional step provides for visualization and/or imaging of the distribution and localization of the optical agent prior to photoactivation of the photosensitizer component, that is useful for accessing highly localized delivery of phototherapeutic treatment.

### 2c Methods for Imaging and Visualization Using Dithienofuran Compounds

In general, molecules absorbing, emitting, or scattering in the visible or NIR region of the electromagnetic spectrum are useful for optical measurement. The high sensitivity associated with fluorescence permits detection without the negative effects of radioactivity or ionizing radiation. Some compounds of the invention absorb strongly in the visible and/or NIR regions. Furthermore, the electronic properties of these systems are very sensitive to substitution patterns in rings of the dithienofuran dye compound and allows for “tuning” the absorption and emission properties using the information described herein.

In an embodiment of this aspect, the invention provides a method of using an optical agent, for example, in a biomedical procedure for optically imaging or visualizing a target tissue or a class of target tissues. The present methods include tissue selective imaging and visualization methods, such as imaging or visualization of renal tissue. A method of this aspect comprises the step of administering a diagnostically effective amount of a compound to a subject, wherein the compound is a compound having any of formulae (FX1) to (FX4) or a pharmaceutical preparation thereof. The present methods are useful for imaging or visualizing colorectal cancer and other cancers, including prostate cancer, gastric cancer, esophageal cancer, uterine-endometrial cancer, pancreatic cancer, breast cancer, cervical cancer, head and neck cancer, hepatic cancer, skin cancer, gallbladder cancer, ling cancer and ovarian cancer.

In methods of this aspect, the compound that has been administered to the subject then is exposed in vivo to electromagnetic radiation and electromagnetic radiation emitted or scattered by the compound is then detected. In some embodiments, fluorescence is excited from the compound (e.g., due to the electromagnetic radiation exposure), optionally via multiphoton excitation processes. In an embodiment particularly useful for imaging and/or visualization, the method of this aspect further comprises: (i) exposing a compound, such as a compound having any one of formula (FX1) to (FX4), administered to the subject to electromagnetic radiation for exciting emission from the compound; and (ii) measuring the emission from the compound administered to the subject. In some embodiments, the methods of the present invention use fluorescence excitation via exposure to light having wavelengths selected over the range of 400-1300 nm. For example, optical coherence tomography (OCT) is an optical imaging technique compatible with the present compounds that allows high resolution cross sectional imaging of tissue microstructure. OCT methods use wavelengths of about 1280 nm. Use of electromagnetic radiation having wavelengths selected over the range of 700 nanometers to 1300 nanometers may be useful for some in situ optical imaging methods of the present invention, including biomedical applications for imaging organs, tissue and/or tumors, anatomical visualization, optical guided surgery and endoscopic procedures. Compounds in present methods may function as contrast agents, optical probes and/or tracer elements. The methods of the present invention include in vivo, in vitro and ex vivo imaging and visualization. The present invention provides methods for a range of clinical procedures, including optical imaging methods and/or visualization guided surgery and/or endoscopic diagnostic and therapeutic procedures.

In an exemplary protocol of uses of the compounds of the invention for a biomedical imaging procedure, the dithienofuran dye is exposed to visible and/or near infrared light. This exposure of the dithienofuran dye to light may occur at any appropriate time but preferably occurs while the dithienofuran dye is located in the body. Due to this exposure of the dithienofuran dye to the visible and/or infrared light, the dithienofuran dye emits spectral energy (e.g., visible and/or near infrared light) that may be detected by appropriate detection equipment. The spectral energy emitted from the dithienofuran dye tends to exhibit a wavelength range greater than a wavelength range absorbed by the dithienofuran dye. For example, if the dithienofuran dye absorbs light of about 700 nm, the dithienofuran dye may emit light of about 745 nm.

Detection of the dithienofuran dye (e.g., light emitted therefrom) may be achieved through optical fluorescence, absorbance or light scattering procedures known in the art. This detection of a portion of the emitted spectral energy, or luminescence, may be characterized as a collection of the emitted spectral energy and a generation of electrical signals indicative of the collected spectral energy. For these purposes, the term “luminescence” refers to the emission of light from excited electronic states of atoms or molecules. Luminescence generally refers to light emission, such as photoluminescence, chemiluminescence, and electrochemiluminescence, among others. In photoluminescence, including fluorescence and phosphorescence, the excited electronic state is created by the absorption of electromagnetic radiation. Luminescence detection involves detection of one or more properties of the luminescence or associated luminescence process. These properties may include intensity, excitation and/or emission wavelength or spectrum, polarization, lifetime, and energy transfer, among others. These properties may also include time-independent (steady-state) and/or time-dependent (time-resolved) properties of the luminescence. Representative luminescence techniques include fluorescence intensity (FLINT), fluorescence polarization (FP), fluorescence resonance energy transfer (FRET), fluorescence lifetime (FLT), total internal reflection fluorescence (TIRF), fluorescence correlation spectroscopy (FCS), fluorescence recovery after photobleaching (FRAP), optical-acoustic tomography (OAT) and bioluminescence resonance energy transfer (BRET), and multiphoton technology, among others.

By way of example, when a compound is used in the present invention, it is desirable that the wavelength of light supplied to the compound be such that it excites the compound. This excitation causes the molecule to emit part of the absorbed energy at a different wavelength, and the emission can be detected using fluorometric techniques or other techniques as described above. One skilled in the art can readily determine the most appropriate detection technique based on, in part, the specific compound(s) administered, the particular use (e.g., tissue to be detected) and other aspects, including physical limitations of the analysis.

The techniques utilized to detect the spectral energy from the dithienofuran dye that is present in the body may be designed to detect only selected wavelengths (or wavelength ranges) and/or may include one or more appropriate spectral filters. Various catheters, endoscopes, ear clips, headbands, surface coils, finger probes, and the like may be utilized to expose the dithienofuran dye to light and/or to detect light emitting therefrom. This detection of spectral energy may be accomplished at one or more times intermittently or may be substantially continuous.

Preferably, non-ionizing energy is administered to the subject or sample for detecting or imaging a biological sample to a compound of the invention. For these purposes, the term “non-ionizing energy” generally refers to electromagnetic radiation wherein a single photon does not carry enough energy to completely remove at least one electron from an atom or molecule of the patient's body. For example, in some embodiments, non-ionizing energy may include spectral energy ranging in wavelength from about 400 nm to about 1300 nm. In some embodiments, non-ionizing energy may simply include visible and/or near infrared light.

In an aspect, the present invention provides an optical imaging method. A method comprises (i) administering an effective amount of an optical agent of the present invention to a subject (e.g., a patient undergoing treatment or diagnosis), for example an optical agent being of formulae (FX1)-(FX4). In this aspect, the optical agent comprises a dithienofuran dye of the present invention, optionally having a targeting ligand and/or photosensitizer component(s). Electromagnetic radiation transmitted, scattered or emitted by the optical agent is then detected. In some embodiments, fluorescence may be excited from the optical agent (e.g., due to the electromagnetic radiation exposure), optionally via multiphoton excitation processes. In some embodiments, the methods of the present invention use fluorescence excitation via exposure to light having wavelengths selected over the range of 300-1300 nm. For example, optical coherence tomography (OCT) is an optical imaging technique compatible with the present optical agents that allows high resolution cross sectional imaging of tissue microstructure. OCT methods use wavelengths of about 1280 nm. Use of electromagnetic radiation having wavelengths selected over the range of 700 nanometers to 1300 nanometers may be useful for some in situ optical imaging methods of the present invention, including biomedical applications for imaging organs, tissue and/or tumors, anatomical visualization, optical guided surgery and endoscopic procedures. This aspect of the present invention can be used for the detection of tumors such as small micrometastases of, e.g., somatostatin subtype 2 (SST-2) positive tumors, and for the identification, characterization and diagnosis of atherosclerotic plaques and blood clots.

In an embodiment particularly useful for imaging and/or visualization the method of this aspect further comprises: (i) exposing a detectable agent, such as an optical agent having any one of formula (FX1)-(FX4), administered to the subject to electromagnetic radiation for exciting emission from the detectable agent; (ii) measuring the emission from the detectable agent, and (iii) optionally generating an image of the emission from the optical agent in the subject. In some embodiments wherein a targeted optical agent is administered to the subject, generating an image of emission from the optical agent allows for visualization of a target tissue. Optionally, methods of this aspect may include site specific delivery of the detectable agent to one or more selected tissue, organ or cell types of the patient, for example by administration of an optical agent having targeting or molecular recognition functionality. Optical agents in present methods may function as contrast agents, optical probes and/or tracer elements. The methods of the present invention include in vivo, in vitro and ex vivo imaging and visualization. The present invention provides methods for a range of clinical procedures, including optical image and/or visualization guided surgery and/or endoscopic diagnostic and therapeutic procedures.

2.d. Biotargeting Using Dithienofuran Compounds

Compounds of the invention are also useful for targeting selected biological materials and/or environments (e.g., cells, tissue, organs, tumors, lesions, etc.). Targeted moieties may also undergo subsequent or coincident phototherapeutic or photodiagnostic applications.

In aspects of this embodiment, compounds of the formulas (FX1) to (FX4) contain one or more biotargeting groups. By way of example, the dithienofuran compound which includes a targeting moiety can be administered to a patient in a diagnostically effective amount to detect the dithienofuran compound within the patient. After a period of time has lapsed for the compound to bind to, or otherwise associate with, the desired target, the whole body or portion thereof is exposed to light of suitable wavelength to excite the dithienofuran compound. Light emanating from the patient as a result of the absorption and excitation of the dithienofuran compound is then detected. By evaluating the location and strength of light emanating from the patient, a diagnosis, prognosis or other assessment can be made as a result of the targeting properties of the dithienofuran compound.

In embodiments, compounds of the invention are useful for both oncology and non-oncology applications. Some specific targets are tumors accessible via endoscope. In this application, a compound that targets a peptide associated with such a tumor is administered to the tumor via endoscope or other useful method. Then, the compounds of the invention can be used in phototherapeutic applications or imaging applications. Other specific targets include colon, lung, ovarian, cervical, esophageal, bladder, blood, and stomach cancers; endometriosis, and bacterial infections. Particular targeting groups include ST receptor binding agents, bombesin receptor binding agents, leukemia peptides, and folate receptor binding. Some examples of targeting peptides are described in PCT Publication no. WO/2008/108941 having a publication date of Dec. 9, 2009 and corresponding to PCT international application no PCT/US2008/002463.

### EXAMPLE 3

### Pharmaceutical Formulations

In an embodiment, the invention provides a pharmaceutical formulation comprising a composition of the invention, such as a compound of any one of formulae (FX1)-(FX4). In an embodiment, the invention provides a method of synthesizing a composition of the invention or a pharmaceutical formulation thereof, such as a compound of any one of formulae (FX1)-(FX4). In an embodiment, a pharmaceutical formulation comprises one or more excipients, carriers, diluents, and/or other components as would be understood in the art. Preferably, the components meet the standards of the National Formulary (“NF”), United States Pharmacopoeia (“USP”; United States Pharmacopeia Convention Inc., Rockville, Md.), or Handbook of Pharmaceutical Manufacturing Formulations (Sarfaraz K. Niazi, all volumes, ISBN: 9780849317521, ISBN 10: 0849317525; CRC Press, 2004). See, e.g., United States Pharmacopeia and National Formulary (USP 30-NF 25), Rockville, Md.: United States Pharmacopeial Convention; 2007; and 2008, and each of any earlier editions; The Handbook of Pharmaceutical Excipients, published jointly by the American Pharmacists Association and the Pharmaceutical Press (Pharmaceutical Press (2005) (ISBN-10: 0853696187, ISBN-13: 978-0853696186); Merck Index, Merck & Co., Rahway, N.J.; and Gilman et al., (eds) (1996); Goodman and Gilman's: The Pharmacological Bases of Therapeutics, 8th Ed., Pergamon Press. In embodiments, the formulation base of the formulations of the invention comprises physiologically acceptable excipients, namely, at least one binder and optionally other physiologically acceptable excipients. Physiologically acceptable excipients are those known to be usable in the pharmaceutical technology sectors and adjacent areas, particularly, those listed in relevant pharmacopeias (e.g. DAB, Ph. Eur., BP, NF, USP), as well as other excipients whose properties do not impair a physiological use.

In an embodiment, an effective amount of a composition of the invention is a therapeutically effective amount. In an embodiment, an effective amount of a composition of the invention is a diagnostically effective amount. In an embodiment, an active ingredient or other component is included in a therapeutically acceptable amount. In an embodiment, an active ingredient or other component is included in a diagnostically acceptable amount.

Variations on compositions including salts and ester forms of compounds: Compounds of this invention and compounds useful in the methods of this invention include those of the compounds and formula(s) described herein and pharmaceutically-acceptable salts and esters of those compounds. In embodiments, salts include any salts derived from the acids and bases of the formulas herein which acceptable for use in human or veterinary applications. In embodiments, the term esters refers to hydrolyzable esters of compounds of the names and structural formulas herein. In embodiments, salts and esters of the compounds of the formulas herein can include those which have the same or better therapeutic, diagnostic, or pharmaceutical (human or veterinary) general properties as the compounds of the formulas herein. In an embodiment, a composition of the invention is a compound or salt or ester thereof suitable for pharmaceutical formulations.

In an embodiment, the invention provides a method for treating a medical condition comprising administering to a subject (e.g. patient) in need thereof, a therapeutically effective amount of a composition of the invention, such as a compound of any one of formulae (FX1)-(FX4). In an embodiment, the medical condition is cancer, or various other diseases, injuries, and disorders, including cardiovascular disorders such as atherosclerosis and vascular restenosis, inflammatory diseases, ophthalmic diseases and dermatological diseases.

In an embodiment, the invention provides a medicament which comprises a therapeutically effective amount of one or more compositions of the invention, such as a compound of any one of formulae (FX1)-(FX4). In an embodiment, the invention provides a medicament which comprises a therapeutically or diagnostically effective amount of one or more compositions of the invention. In an embodiment, the invention provides a method for making a medicament for treatment of a condition described herein. In an embodiment, the invention provides a method for making a medicament for diagnosis or aiding in the diagnosis of a condition described herein. In an embodiment, the invention provides the use of one or more compositions set forth herein for the making of a medicament.

Compounds of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds set forth herein.

In an embodiment, a composition of the invention is isolated or purified. In an embodiment, an isolated or purified compound may be at least partially isolated or purified as would be understood in the art.

Typically, a compound of the present invention, or pharmaceutically acceptable salt thereof, is administered to a subject in a diagnostically or therapeutically effective amount. One skilled in the art generally can determine an appropriate dosage. Factors affecting a particular dosage regimen (including the amount of compound delivered, frequency of administration, and whether administration is continuous or intermittent) include, for example, the type, age, weight, sex, diet, and condition of the subject; the type of pathological condition and its severity; and the nature of the desired effect. Pharmacological considerations include dithienofuran compound activity, efficacy, pharmacokinetic, and toxicology profiles of the particular dithienofuran compound used; the route of administration and whether a drug delivery system is utilized; and whether the dithienofuran compound is administered as part of a combination therapy (e.g., whether the agent is administered in combination with one or more active compounds, other agents, radiation, and the like).

Compositions for oral administration may be, for example, prepared in a manner such that a single dose in one or more oral preparations contains at least about 20 mg of the dithienofuran compound per square meter of subject body surface area, or at least about 50, 100, 150, 200, 300, 400, or 500 mg of the dithienofuran compound per square meter of subject body surface area (the average body surface area for a human is, for example, 1.8 square meters). In particular, a single dose of a composition for oral administration can contain from about 20 to about 600 mg, and in certain aspects from about 20 to about 400 mg, in another aspect from about 20 to about 300 mg, and in yet another aspect from about 20 to about 200 mg of the dithienofuran compound per square meter of subject body surface area. Compositions for parenteral administration can be prepared in a manner such that a single dose contains at least about 20 mg of the dithienofuran compound per square meter of subject body surface area, or at least about 40, 60, 100, 150, 200, 300, 400, or 500 mg of the dithienofuran compound per square meter of subject body surface area. In particular, a single dose in one or more parenteral preparations contains from about 20 to about 500 mg, and in certain aspects from about 20 to about 400, and in another aspect from about 20 to about 400 mg, and in yet another aspect from about 20 to about 350 mg of the dithienofuran compound per square meter of subject body surface area. It should be recognized that these oral and parenteral dosage ranges represent generally preferred dosage ranges, and are not intended to limit the invention. The dosage regimen actually employed can vary widely, and, therefore, can deviate from the generally preferred dosage regimen. It is contemplated that one skilled in the art will tailor these ranges to the individual subject.

As indicated above, it is contemplated that the compounds and pharmaceutically acceptable salts of the present invention may be used as part of a combination. The term “combination” means the administration of two or more compounds directed to the target condition. The treatments of the combination generally may be co-administered in a simultaneous manner. Two compounds can be co-administered as, for example: (a) a single formulation (e.g., a single capsule) having a fixed ratio of active ingredients; or (b) multiple, separate formulations (e.g., multiple capsules) for each compound. The treatments of the combination may alternatively (or additionally) be administered at different times.

It is further contemplated that the dithienofuran compounds and salts of this invention can be used in the form of a kit that is suitable for use in performing the methods described herein, packaged in a container. The kit can contain the dithienofuran compound or compounds and, optionally, appropriate diluents, devices or device components suitable for administration and instructions for use in accordance with the methods of the present invention. The devices can include parenteral injection devices, such as syringes or transdermal patch or the like. Device components can include cartridges for use in injection devices and the like. In one aspect, the kit includes a first dosage form including a dithienofuran compound or salt of this invention and a second dosage form including another active ingredient in quantities sufficient to carry out the methods of the present invention. The first dosage form and the second dosage form together can include a therapeutically effective amount of the compounds for treating the targeted condition(s).

This invention also is directed, in part, to pharmaceutical compositions including a therapeutically effective amount of a compound or salt of this invention, as well as processes for making such compositions. Such compositions generally include one or more pharmaceutically acceptable carriers (e.g., excipients, vehicles, auxiliaries, adjuvants, diluents) and may include other active ingredients. Formulation of these compositions may be achieved by various methods known in the art. A general discussion of these methods may be found in, for example, Hoover, John E., Remington's Pharmaceutical Sciences (Mack Publishing Co., Easton, Pa.: 1975). See also, Lachman, L., eds., Pharmaceutical Dosage Forms (Marcel Decker, New York, N.Y., 1980).

The preferred composition depends on the route of administration. Any route of administration may be used as long as the target of the compound or pharmaceutically acceptable salt is available via that route. Suitable routes of administration include, for example, oral, parenteral, inhalation, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual, and intestinal administration.

Pharmaceutically acceptable carriers that may be used in conjunction with the compounds of the invention are well known to those of ordinary skill in the art. Carriers can be selected based on a number of factors including, for example, the particular dithienofuran compound(s) or pharmaceutically acceptable salt(s) used; the compound's concentration, stability, and intended bioavailability; the condition being treated; the subject's age, size, and general condition; the route of administration; etc. A general discussion related to carriers may be found in, for example, J. G. Naim, Remington's Pharmaceutical Science, pp. 1492-1517 (A. Gennaro, ed., Mack Publishing Co., Easton, Pa. (1985)).

Solid dosage forms for oral administration include, for example, capsules, tablets, gelcaps, pills, dragees, troches, powders, granules, and lozenges. In such solid dosage forms, the compounds or pharmaceutically acceptable salts thereof can be combined with one or more pharmaceutically acceptable carriers. The compounds and pharmaceutically acceptable salts thereof can be mixed with carriers including, but not limited to, lactose, sucrose, starch powder, corn starch, potato starch, magnesium carbonate, microcrystalline cellulose, cellulose esters of alkanoic acids, cellulose alkyl esters, talc, stearic acid, magnesium stearate, magnesium oxide, sodium and calcium salts of phosphoric and sulfuric acids, sodium carbonate, agar, mannitol, sorbitol, sodium saccharin, gelatin, acacia gum, alginic acid, sodium alginate, tragacanth, colloidal silicon dioxide, croscarmellose sodium, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can include buffering agents, such as sodium citrate, or magnesium or calcium carbonate or bicarbonate. Tablets and pills additionally can, for example, include a coating (e.g., an enteric coating) to delay disintegration and absorption. The concentration of the dithienofuran compound in a solid oral dosage form can be from about 5 to about 50%, and in certain aspects from about 8 to about 40%, and in another aspect from about 10 to about 30% by weight based on the total weight of the composition.

Liquid dosage forms of the compounds of the present invention for oral administration include, for example, pharmaceutically acceptable emulsions, solutions, suspensions, syrups, and elixirs containing inert diluents commonly used in the art (e.g., water). Such compositions also can include adjuvants, such as wetting, emulsifying, suspending, flavoring (e.g., sweetening), and/or perfuming agents. The concentration of the dithienofuran compound in the liquid dosage form can be from about 0.01 to about 5 mg, and in certain aspects from about 0.01 to about 1 mg, and in another aspect from about 0.01 to about 0.5 mg per ml of the composition. Low concentrations of the compounds of the present invention in liquid dosage form can be prepared in the case that the dithienofuran compound is more soluble at low concentrations. Techniques for making oral dosage forms useful in the present invention are generally described in, for example, Modern Pharmaceutics, Chapters 9 and 10 (Banker & Rhodes, Editors (1979)). See also, Lieberman et al., Pharmaceutical Dosage Forms: Tablets (1981). See also, Ansel, Introduction to Pharmaceutical Dosage Forms (2nd Edition (1976)).

In some aspects of the present invention, tablets or powders for oral administration can be prepared by dissolving the dithienofuran compound in a pharmaceutically acceptable solvent capable of dissolving the compound to form a solution and then evaporating when the solution is dried under vacuum. A carrier can also be added to the solution before drying. The resulting solution can be dried under vacuum to form a glass. The glass can then mix with a binder to form a powder. This powder may be mixed with fillers or other conventional tableting agents, and then processed to form a tablet. Alternatively, the powder may be added to a liquid carrier to form a solution, emulsion, suspension, or the like.

In some aspects, solutions for oral administration are prepared by dissolving the dithienofuran compound in a pharmaceutically acceptable solvent capable of dissolving the compound to form a solution. An appropriate volume of a carrier is added to the solution while stirring to form a pharmaceutically acceptable solution for oral administration.

“Parenteral administration” includes subcutaneous injections, intravenous injections, intraarterial injections, intraorbital injections, intracapsular injections, intraspinal injections, intraperitoneal injections, intramuscular injections, intrasternal injections, and infusion. Dosage forms suitable for parenteral administration include solutions, suspensions, dispersions, emulsions, and any other dosage form that can be administered parenterally.

Injectable preparations (e.g., sterile injectable aqueous or oleaginous suspensions) can be formulated according to the known art using suitable dispersing, wetting agents, and/or suspending agents. Acceptable vehicles for parenteral use include both aqueous and nonaqueous pharmaceutically-acceptable solvents. Suitable pharmaceutically acceptable aqueous solvents include, for example, water, saline solutions, dextrose solutions (e.g., such as DW5), electrolyte solutions, etc.

In one embodiment, the present dithienofuran compounds are formulated as nanoparticles or microparticles. Use of such nanoparticle or microparticle formulations may be beneficial for some applications to enhance delivery, localization, target specificity, administration, etc. of the dithienofuran compound. Potentially useful nanoparticles and microparticles include, but are not limited to, micelles, liposomes, microemulsions, nanoemulsions, vesicles, tubular micelles, cylindrical micelles, bilayers, folded sheets structures, globular aggregates, swollen micelles, inclusion complex, encapsulated droplets, microcapsules, nanocapsules or the like. As will be understood by those having skill in the art, the present dithienofuran compounds can be located inside the nanoparticle or microparticle, within a membrane or wall of the nanoparticle or microparticle, or outside of (but bonded to or otherwise associated with) the nanoparticle or microparticle. The agent formulated in nanoparticles or microparticles may be administered by any of the routes previously described. In a formulation applied topically, the dithienofuran compound is slowly released over time. In an injectable formulation, the liposome, micelle, capsule, etc., circulates in the bloodstream and is delivered to the desired site (e.g., target tissue).

Preparation and loading of nanoparticles and microparticles are well known in the art. As one example, liposomes may be prepared from dipalmitoyl phosphatidylcholine (DPPC) or egg phosphatidylcholine (PC) because this lipid has a low heat transition. Liposomes are made using standard procedures as known to one skilled in the art (e.g., Braun-Falco et al., (Eds.), Griesbach Conference, Liposome Dermatics, Springer-Verlag, Berlin (1992), pp. 69 81; 91 117 which is expressly incorporated by reference herein). Polycaprolactone, poly(glycolic) acid, poly(lactic) acid, polyanhydride or lipids may be formulated as microspheres. As an illustrative example, the present dithienofuran compounds may be mixed with polyvinyl alcohol (PVA), the mixture then dried and coated with ethylene vinyl acetate, then cooled again with PVA. In a liposome, the present dithienofuran compounds may be within one or both lipid bilayers, in the aqueous between the bilayers, or with the center or core. Liposomes may be modified with other molecules and lipids to form a cationic liposome. Liposomes may also be modified with lipids to render their surface more hydrophilic which increases their circulation time in the bloodstream. The thus-modified liposome has been termed a “stealth” liposome, or a long-lived liposome, as described in U.S. Pat. No. 6,258,378, and in Stealth Liposomes, Lasic and Martin (Eds.) 1995 CRC Press, London, which are expressly incorporated by reference herein. Encapsulation methods include detergent dialysis, freeze drying, film forming, injection, as known to one skilled in the art and disclosed in, for example, U.S. Pat. No. 6,406,713 which is expressly incorporated by reference herein in its entirety.

Suitable pharmaceutically-acceptable nonaqueous solvents include, but are not limited to, the following (as well as mixtures thereof): alcohols (these include, for example, σ-glycerol formal, β-glycerol formal, 1,3-butyleneglycol, aliphatic or aromatic alcohols having from 2 to about 30 carbons (e.g., methanol, ethanol, propanol, isopropanol, butanol, t-butanol, hexanol, octanol, amylene hydrate, benzyl alcohol, glycerin (glycerol), glycol, hexylene, glycol, tetrahydrofuranyl alcohol, cetyl alcohol, and stearyl alcohol), fatty acid esters of fatty alcohols (e.g., polyalkylene glycols, such as polypropylene glycol and polyethylene glycol), sorbitan, sucrose, and cholesterol); amides (these include, for example, dimethylacetamide (DMA), benzyl benzoate DMA, dimethylformamide, N-hydroxyethyO-lactamide, N,N-dimethylacetamide-amides, 2-pyrrolidinone, 1-methyl-2-pyrrolidinone, and polyvinylpyrrolidone); esters (these include, for example, acetate esters (e.g., monoacetin, diacetin, and triacetin), aliphatic and aromatic esters (e.g., ethyl caprylate or octanoate, alkyl oleate, benzyl benzoate, or benzyl acetate), dimethylsulfoxide (DMSO), esters of glycerin (e.g., mono, di, and tri-glyceryl citrates and tartrates), ethyl benzoate, ethyl acetate, ethyl carbonate, ethyl lactate, ethyl oleate, fatty acid esters of sorbitan, glyceryl monostearate, glyceride esters (e.g., mono, di, or tri-glycerides), fatty acid esters (e.g., isopropyl myristrate), fatty acid derived PEG esters (e.g., PEG-hydroxyoleate and PEG-hydroxystearate), N-methyl pyrrolidinone, pluronic 60, polyoxyethylene sorbitol oleic polyesters (e.g., poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate), polyoxyethylene sorbitan esters (e.g., polyoxyethylene-sorbitan monooleate, polyoxyethylene-sorbitan monopalmitate, polyoxyethylene-sorbitan monolaurate, polyoxyethylene-sorbitan monostearate, and POLYSORBATE 20, 40, 60, and 80 (from ICI Americas, Wilmington, Del.)), polyvinylpyrrolidone, alkyleneoxy modified fatty acid esters (e.g., polyoxyl 40 hydrogenated castor oil and polyoxyethylated castor oils, such as CREMOPHOR EL solution or CREMOPHOR RH 40 solution), saccharide fatty acid esters (i.e., the condensation product of a monosaccharide (e.g., pentoses, such as, ribose, ribulose, arabinose, xylose, lyxose, and xylulose; hexoses, such as glucose, fructose, galactose, mannose, and sorbose; trioses; tetroses; heptoses; and octoses), disaccharide (e.g., sucrose, maltose, lactose, and trehalose), oligosaccharide, or a mixture thereof with one or more C₄-C₂₂ fatty acids (e.g., saturated fatty acids, such as caprylic acid, capric acid, lauric acid, myristic acid, palmitic acid, and stearic acid; and unsaturated fatty acids, such as palmitoleic acid, oleic acid, elaidic acid, erucic acid, and linoleic acid), and steroidal esters); ethers (these are typically alkyl, aryl, and cyclic ethers having from 2 to about 30 carbons. Examples include diethyl ether, tetrahydrofuran, dimethyl isosorbide, diethylene glycol monoethyl ether), and glycofurol (tetrahydrofurfuranyl alcohol polyethylene glycol ether); ketones (these typically have from about 3 to about 30 carbons. Examples include acetone, methyl ethyl ketone, methyl isobutyl ketone); hydrocarbons (these are typically aliphatic, cycloaliphatic, and aromatic hydrocarbons having from about 4 to about 30 carbons). Examples include benzene, cyclohexane, dichloromethane, dioxolanes, hexane, n-decane, n-dodecane, n-hexane, sulfolane, tetramethylenesulfone, tetramethylenesulfoxide, toluene, dimethylsulfoxide (DMSO); and tetramethylene sulfoxide; oils (these include oils of mineral, vegetable, animal, essential, or synthetic origin). These include mineral oils, such as aliphatic and wax-based hydrocarbons, aromatic hydrocarbons, mixed aliphatic and aromatic based hydrocarbons, and refined paraffin oil; vegetable oils, such as linseed, tung, safflower, soybean, castor, cottonseed, groundnut, rapeseed, coconut, palm, olive, corn, corn germ, sesame, persic, and peanut oil; glycerides, such as mono-, di-, and triglycerides; animal oils, such as fish, marine, sperm, cod-liver, haliver, squaiene, squalane, and shark liver oil; oleic oils; and polyoxyethylated castor oil); alkyl, alkenyl, or aryl halides (these include alkyl or aryl halides having from 1 to about 30 carbons and one or more halogen substituents. Examples include methylene chloride); monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (SOLUTOL HS-15, from BASF, Ludwigshafen, Germany); polyoxyethylene glycerol; sodium laurate; sodium oleate; and sorbitan monooleate. Other pharmaceutically acceptable solvents for use in the invention are well known to those of ordinary skill in the art. General discussion relating to such solvents may be found in, for example, The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics 3d ed., (G. Banker et. al., eds., Marcel Dekker, Inc., New York, N.Y. (1995)), The Pharmacological Basis of Therapeutics, (Goodman & Gilman, McGraw Hill Publishing), Pharmaceutical Dosage Forms, (H. Lieberman et. al., eds., Marcel Dekker, Inc., New York, New York (1980)), Remington's Pharmaceutical Sciences, 19th ed., (A. Gennaro, ed., Mack Publishing, Easton, Pa., (1995)), The United States Pharmacopeia 24, The National Formulary 19, (National Publishing, Philadelphia, Pa. (2000)); Spiegel, A. J., et al., “Use of Nonaqueous Solvents in Parenteral Products,” J. Pharma. Sciences, Vol. 52, No. 10, pp. 917-927 (1963).

Solvents useful in the present invention include, but are not limited to, those known to stabilize the dithienofuran compounds or pharmaceutically acceptable salts thereof. These typically include, for example, oils rich in triglycerides, such as safflower oil, soybean oil, and mixtures thereof; and alkyleneoxy-modified fatty acid esters, such as polyoxyl 40 hydrogenated castor oil and polyoxyethylated castor oils (e.g., CREMOPHOR EL solution or CREMOPHOR RH 40 solution). Commercially available triglycerides include INTRALIPID emulsified soybean oil (Kabi-Pharmacia Inc., Stockholm, Sweden), NUTRALIPID emulsion (McGaw, Irvine, Calif.), LIPOSYN II 20% emulsion (a 20% fat emulsion solution containing 100 mg safflower oil, 100 mg soybean oil, 12 mg egg phosphatides, and 25 mg glycerin per ml of solution; Abbott Laboratories, Chicago, Ill.), LIPOSYN III 2% emulsion (a 2% fat emulsion solution containing 100 mg safflower oil, 100 mg soybean oil, 12 mg egg phosphatides, and 25 mg glycerin per ml of solution; Abbott Laboratories, Chicago, Ill.), natural or synthetic glycerol derivatives containing the docosahexaenoyl group at levels of from about 25 to about 100% (by weight based on the total fatty acid content) (DHASCO from Martek Biosciences Corp., Columbia, Md.; DHA MAGURO from Daito Enterprises, Los Angeles, Calif.; SOYACAL; and TRAVEMULSION). Ethanol in particular is a useful solvent for dissolving a dithienofuran compound or pharmaceutically acceptable salt thereof to form solutions, emulsions, and the like.

Additional components can be included in the compositions of this invention for various purposes generally known in the pharmaceutical industry. These components tend to impart properties that, for example, enhance retention of the dithienofuran compound or salt at the site of administration, protect the stability of the composition, control the pH, and facilitate processing of the dithienofuran compound or salt into pharmaceutical formulations, and the like. Specific examples of such components include cryoprotective agents; agents for preventing reprecipitation of the dithienofuran compound or salt surface; active, wetting, or emulsifying agents (e.g., lecithin, polysorbate-80, TWEEN 80, pluronic 60, and polyoxyethylene stearate); preservatives (e.g., ethyl-p-hydroxybenzoate); microbial preservatives (e.g., benzyl alcohol, phenol, m-cresol, chlorobutanol, sorbic acid, thimerosal, and paraben); agents for adjusting pH or buffering agents (e.g., acids, bases, sodium acetate, sorbitan monolaurate, etc.); agents for adjusting osmolarity (e.g., glycerin); thickeners (e.g., aluminum monostearate, stearic acid, cetyl alcohol, stearyl alcohol, guar gum, methyl cellulose, hydroxypropylcellulose, tristearin, cetyl wax esters, polyethylene glycol, etc.); colorants; dyes; flow aids; non-volatile silicones (e.g., cyclomethicone); clays (e.g., bentonites); adhesives; bulking agents; flavorings; sweeteners; adsorbents; fillers (e.g., sugars such as lactose, sucrose, mannitol, sorbitol, cellulose, calcium phosphate, etc.); diluents (e.g., water, saline, electrolyte solutions, etc.); binders (e.g., gelatin; gum tragacanth; methyl cellulose; hydroxypropyl methylcellulose; sodium carboxymethyl cellulose; polyvinylpyrrolidone; sugars; polymers; acacia; starches, such as maize starch, wheat starch, rice starch, and potato starch; etc.); disintegrating agents (e.g., starches, such as maize starch, wheat starch, rice starch, potato starch, and carboxymethyl starch; cross-linked polyvinyl pyrrolidone; agar; alginic acid or a salt thereof, such as sodium alginate; croscarmellose sodium; crospovidone; etc); lubricants (e.g., silica; talc; stearic acid and salts thereof, such as magnesium stearate; polyethylene glycol; etc.); coating agents (e.g., concentrated sugar solutions including gum arabic, talc, polyvinyl pyrrolidone, carbopol gel, polyethylene glycol, titanium dioxide, etc.); and antioxidants (e.g., sodium metabisulfite, sodium bisulfite, sodium sulfite, dextrose, phenols, thiophenols, etc.). Techniques and compositions for making parenteral dosage forms are generally known in the art. Formulations for parenteral administration may be prepared from one or more sterile powders and/or granules having a compound or salt of this invention and one or more of the carriers or diluents mentioned for use in the formulations for oral administration. The powder or granule typically is added to an appropriate volume of a solvent (typically while agitating (e.g., stirring) the solvent) that is capable of dissolving the powder or granule. Particular solvents useful in the invention include, for example, water, polyethylene glycol, propylene glycol, ethanol, corn oil, cottonseed oil, peanut oil, sesame oil, benzyl alcohol, sodium chloride, and/or various buffers.

Emulsions for parenteral administration can be prepared by, for example, dissolving a compound or salt of this invention in any pharmaceutically acceptable solvent capable of dissolving the compound to form a solution; and adding an appropriate volume of a carrier, which is an emulsion, to the solution while stirring to form the emulsion. Solutions for parenteral administration can be prepared by, for example, dissolving a compound or salt of this invention in any pharmaceutically acceptable solvent capable of dissolving the compound to form a solution; and adding an appropriate volume of a carrier to the solution while stirring to form the solution.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, cocoa butter; synthetic mono-, di-, or triglycerides; fatty acids; and/or polyethylene glycols.

“Topical administration” includes the use of transdermal administration, such as transdermal patches or iontophoresis devices.

If desired, the emulsions or solutions described above for oral or parenteral administration can be packaged in IV bags, vials, or other conventional containers in concentrated form, and then diluted with a pharmaceutically acceptable liquid (e.g., saline) to form an acceptable dithienofuran concentration before use.

Other adjuvants and modes of administration well known in the pharmaceutical art may also be used. Pharmaceutically acceptable salts comprise pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R¹)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl³¹ , Br⁻), sulfate, acetates (e.g., acetate, trifluoroacetate), ascorbates, aspartates, benzoates, citrates, and lactate.

It is understood that this invention is not limited to the particular compounds, methodology, protocols, and reagents described, as these may vary. It is also to be understood that the terminology used herein is for the purpose of describing particular embodiments only, and is not intended to limit the scope of the invention which will be limited only by the appended claims.

Compositions of the invention includes formulations and preparations comprising one or more of the present compounds provided in an aqueous solution, such as a pharmaceutically acceptable formulation or preparation. Optionally, compositions of the invention further comprise one or more pharmaceutically acceptable surfactants, buffers, electrolytes, salts, carriers, binders, coatings, preservatives and/or excipients.

### STATEMENTS REGARDING INCORPORATION BY REFERENCE AND VARIATIONS

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Optical agents of the present invention may be formulated with pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R¹)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

The compounds of this invention may contain one or more chiral centers. Accordingly, this invention is intended to include racemic mixtures, diasteromers, enantiomers and mixture enriched in one or more steroisomer. The scope of the invention as described and claimed encompasses the racemic forms of the compounds as well as the individual enantiomers and non-racemic mixtures thereof.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

In certain embodiments, the present invention encompasses administering optical agents useful in the present invention to a patient or subject. A “patient” or “subject', used equivalently herein, refers to an animal. In particular, an animal refers to a mammal, preferably a human. The subject may either: (1) have a condition diagnosable, preventable and/or treatable by administration of an optical agent of the invention; or (2) is susceptible to a condition that is diagnosable, preventable and/or treatable by administering an optical agent of this invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

Compositions of the invention include formulations and preparations comprising one or more of the present optical agents provided in an aqueous formulation, or in a biocompatible, pharmaceutically acceptable biocompatible organic solutions. Optionally, compositions of the present invention further comprise one or more pharmaceutically acceptable surfactants, buffers, electrolytes, salts, carriers and/or excipients.

In some embodiments, a liposome or micelle may be utilized as a carrier or vehicle for the composition. For example, in some embodiments, the dithienofuran dyer may be a part of the lipophilic bilayers or micelle, and the targeting ligand, if present, may be on the external surface of the liposome or micelle. As another example, a targeting ligand may be externally attached to the liposome or micelle after formulation for targeting the liposome or micelle (which contains the inventive dithienofuran dye) to the desired tissue, organ, or other site in the body.

Every formulation or combination of components described or exemplified herein can be used to practice the invention, unless otherwise stated.

The present compositions, preparations and formulations can be used both as a diagnostic agent as well as a phototherapyc agent concomitantly. For example, an effective amount of the present compositions, preparations and formulations in a pharmaceutically acceptable formulation is administered to a patient. Administration is followed by a procedure that combines photodiagnosis and phototherapy. For example, a composition comprising compounds for combined photodiagnosis and phototherapy is administered to a patient and its concentration, localization, or other parameters is determined at the target site of interest. More than one measurement may be taken to determine the location of the target site. The time it takes for the compound to accumulate at the target site depends upon factors such as pharmcokinetics, and may range from about thirty minutes to two days. Once the site is identified, the phototherapeutic part of the procedure may be done either immediately after determining the site or before the agent is cleared from the site. Clearance depends upon factors such as pharmacokinetics.

The present compositions, preparations and formulations can be formulated into diagnostic or therapeutic compositions for enteral, parenteral, topical, aerosol, inhalation, or cutaneous administration. Topical or cutaneous delivery of the compositions, preparations and formulations may also include aerosol formulation, creams, gels, solutions, etc. The present compositions, preparations and formulations are administered in doses effective to achieve the desired diagnostic and/or therapeutic effect. Such doses may vary widely depending upon the particular compositions employed in the composition, the organs or tissues to be examined, the equipment employed in the clinical procedure, the efficacy of the treatment achieved, and the like. These compositions, preparations and formulations contain an effective amount of the composition(s), along with conventional pharmaceutical carriers and excipients appropriate for the type of administration contemplated. These compositions, preparations and formulations may also optionally include stabilizing agents and skin penetration enhancing agents.

Methods of this invention comprise the step of administering an “effective amount” of the present diagnostic and therapeutic compositions, formulations and preparations containing the present compounds, to diagnosis, image, monitor, evaluate treat, reduce or regulate a biological condition and/or disease state in a patient. The term “effective amount,” as used herein, refers to the amount of the diagnostic and therapeutic formulation, that, when administered to the individual is effective diagnosis, image, monitor, evaluate treat, reduce or regulate a biological condition and/or disease state. As is understood in the art, the effective amount of a given composition or formulation will depend at least in part upon, the mode of administration (e.g. intravenous, oral, topical administration), any carrier or vehicle employed, and the specific individual to whom the formulation is to be administered (age, weight, condition, sex, etc.). The dosage requirements need to achieve the “effective amount” vary with the particular formulations employed, the route of administration, and clinical objectives. Based on the results obtained in standard pharmacological test procedures, projected daily dosages of active compound can be determined as is understood in the art.

Any suitable form of administration can be employed in connection with the diagnostic and therapeutic formulations of the present invention. The diagnostic and therapeutic formulations of this invention can be administered intravenously, in oral dosage forms, intraperitoneally, subcutaneously, or intramuscularly, all using dosage forms well known to those of ordinary skill in the pharmaceutical arts.

The diagnostic and therapeutic formulations of this invention can be administered alone, but may be administered with a pharmaceutical carrier selected upon the basis of the chosen route of administration and standard pharmaceutical practice.

The diagnostic and therapeutic formulations of this invention and medicaments of this invention may further comprise one or more pharmaceutically acceptable carrier, excipient, buffer, emulsifier, surfactant, electrolyte or diluent. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985).

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of does not exclude materials or steps that do not materially affect the basic and novel characteristics of the claim. In each instance herein any of the terms “comprising”, “consisting essentially of and “consisting of may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-42. (canceled)

43. A compound being of the formula (FX1): 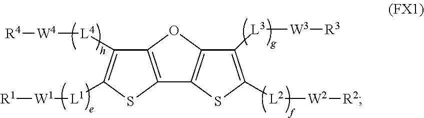
wherein:
each of L¹, L², L³, and L⁴, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;
each of W¹, W², W³, and W⁴ is independently a single bond, —(CH₂)ₙ—, —(HCCH)ₙ, —O—, —S—, —SO—, —SO₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, —NR¹⁸CSNR¹⁹, —O(CH₂)ₙ—, —S(CH₂)ₙ—, —NR²⁰(CH₂)ₙ—, —CO(CH₂)ₙ—, —COO(CH₂)ₙ—, —OCO(CH₂)ₙ—, —OCOO(CH₂)ₙ—, —CONR²¹(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, —NR²⁶CONR²⁷(CH₂)ₙ—, —NR^{2B}CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, or —CO(CH₂)ₙNR³³CO—;
each of R¹, R², R³, and R⁴ is independently hydrogen, —OCF₃, C₁-C₂₀ alkyl, C₅-C₂₀ aryl, C₁-C₂₀ acyl, C₂-C₂₀ alkenyl, C₂⁻C₂₀ alkynyl, C₅-C₂₀ alkylaryl, C₁-C₆ alkoxycarbonyl, halo, halomethyl, dihalomethyl, trihalomethyl, —CO₂R⁴⁰, —SOR⁴¹, —OSR⁴², —SO₂OR⁴³, —CH₂(CH₂OCH₂)_{c}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —CH(R⁶²)CO₂H, —CH(R⁶³)NH₂, —N₃, PS¹, PS², FL, or Bm, wherein at least one of R¹-R⁴ is PS¹ or PS²;
each of a and b is independently an integer selected from the range of 1 to 100;
each n is independently an integer selected from the range of 1 to 10;
each of e, f, g, and h is independently 0 or 1;
each of R¹¹-R³³ is independently hydrogen, C₁-C₂₀ alkyl, or C₅-C₂₀ aryl;
each of R⁴⁰-R⁶¹ is independently hydrogen or C₁-C₁₄ alkyl;
each of R⁶² and R⁶³ is independently a side chain residue of a natural α-amino acid;
each FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound;
each PS¹ is independently a Type 1 photosensitizer;
each PS² is independently a Type 2 photosensitizer; and
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent.

44. The compound of claim 43, wherein at least one of R¹-R⁴ is PS¹.

45. The compound of claim 44, wherein each PS¹ is independently an azide, azo, diazo, oxaza, or diaza group.

46. The compound of claim 43, wherein at least one of R¹-R⁴ is PS².

47. The compound of claim 46, wherein each PS² is independently a porphyrin, benzoporphyrin, phthalocyanine, phenothiazine, chlorin, bacteriochlorin, purpurin, merocyanine, pheophorbides, psoralen, aminolevulinic acid (ALA), hematoporphyrin derivative, porphycene, porphacyanine, cyanine, indocyanine, rhodamine, phenoxazine, a phenoselenazine, fluorescein, squaraine, corrin, croconium, azo dye, methine dye, indolenium dye, halogen, anthracyline, C₁-C₂₀ peroxyalkyl, C₁-C₂₀ peroxyaryl, C₁-C₂₀ sulfenatoalkyl, sulfenatoaryl, naphthalocyanine, methylene blue, or chalcogenopyrylium analogue.

48. The compound of claim 43 being of the formula (FX2):

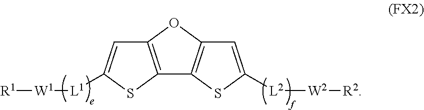

49. The compound of claim 43 being of the formula (FX3):

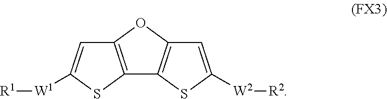

50. The compound of claim 43 being of the formula (FX4):

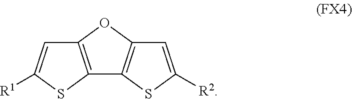

51. The compound of claim 43, wherein at least one of R¹-R⁴ is Bm.

52. The compound of claim 43, wherein:
W¹ is a single bond, —SO—, —SO₂—, or —CO—; and
R¹ is —N₃, —SOR⁴¹, or —OSR⁴².

53. The compound of claim 43, wherein:
W¹ is —O—, —S—, —NR¹¹—, —OCO—, —OCOO—, —NR¹³CO—, —CONR¹²—, —OCONR¹⁴—, or —NR¹⁵COO—;
W² is —SO—, —SO₂—, —SO₃—, —COO—, or —CONR¹²—;
R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL, or Bm; and
R² is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL, or Bm.

54. The compound of claim 43, wherein:
W¹ is —NR¹¹— or —CONR¹²—;
W² is —COO— or —-CONR¹²—;
R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL, or Bm; and
R² is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL, or Bm.

55. The compound of claim 43, wherein at least one of R¹-R⁴ is —OR⁴⁶, —SR⁴⁷, —NR⁴⁸R⁴⁹, or —NR⁵⁶COR⁵¹.

56. The compound of claim 43, wherein at least one of R¹-R⁴ is —CN, halo, —CO₂R⁴⁰, —COR⁵⁴, —NO₂, —SO₂R⁵⁵, or —SO₂NR⁵⁸R⁵⁹.

57. The compound of claim 43, wherein at least one of R¹-R⁴ is —NR⁴⁸R⁴⁹, and at least one of R¹-R⁴ is —CO₂R⁴⁰, —COR⁵⁴, —SO₂NR⁵⁸R⁵⁹, or —SO₂R⁵⁵.

58. A method of using a compound in a medical phototherapy procedure, the method comprising:
administering to a subject in need of treatment a therapeutically effective amount of the compound being of formula (FX1); 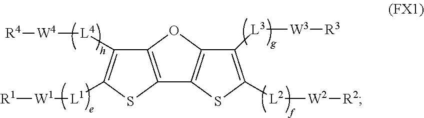
wherein:
each of L¹, L², L³, and L⁴, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;
each of W¹, W², W³, and W⁴ is independently a single bond, —(CH₂)ₙ—, —(HCCH)ₙ—, —O—, —S—, —SO—, —SO₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCO(CH₂)ₙ—, —OCOO(CH₂)ₙ—, —CONR²¹(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, —NR²⁶CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, or —CO(CH₂)ₙNR³³CO—;
each of R¹, R², R³, and R⁴ is independently hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₆ alkoxycarbonyl, halo, halomethyl, dihalomethyl, trihalomethyl, —CO₂R⁴⁰, —SOR⁴¹, —OSR⁴², —SO₂OR⁴³, —CH₂(CH₂OCH₂)_{c}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —CH(R⁶²)CO₂H, —CH(R⁶³)NH₂, —N₃, PS¹, PS², FL, or Bm, wherein at least one of R¹-R⁴ is PS¹ or PS²;
each of a and b is independently an integer selected from the range of 1 to 100;
each n is independently an integer selected from the range of 1 to 10;
each of e, f, g, and h is independently 0 or 1;
each of R¹¹-R³³ is independently hydrogen, C₁-C₂₀ alkyl, or C₅-C₂₀ aryl;
each of R⁴⁰-R⁶¹ is independently hydrogen or C₁-C₁₀ alkyl;
each of R⁶² and R⁶³ is independently a side chain residue of a natural α-amino acid;
each FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound;
each PS¹ is independently a Type 1 photosensitizer;
each PS² is independently a Type 2 photosensitizer;
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent; and
exposing the administered compound to electromagnetic radiation.

59. A method of using a compound in a biomedical procedure for assessing physiological function of an organ or tissue, the method comprising:
administering into a bodily fluid of a subject a diagnostically effective amount of a detectable agent comprising the compound having formula (FM): 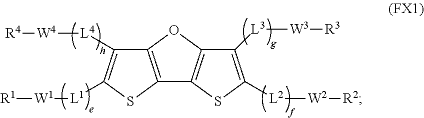
wherein:
each of L¹, L², L³, and L⁴, if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;
each of W¹, W², W³, and W⁴ is independently a single bond, —(CH₂)ₙ—, —(HCCH)ₙ—, —O—, —S—, —SO—, —SO₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, —NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCO(CH₂)ₙ—, —OCOO(CH₂)ₙ—, —CONR²¹(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, —NR²⁶CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, or —CO(CH₂)ₙNR³³CO—;
each of R¹, R², R³, and R⁴ is independently hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₈ alkoxycarbonyl, halo, halomethyl, dihalomethyl, trihalomethyl, —CO₂R⁴⁰, —SOR⁴¹, —OSR⁴², —SO₂OR⁴³, —CH₂(CH₂OCH₂)_{c}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO₃R⁵⁶R⁵⁷, —SO₂NR⁵⁸R⁵⁹, —CH₂(CHOH)ₐR⁶⁰, —(CH₂CH₂O)_{b}R⁶¹, —CH(R⁶²)CO₂H, —CH(R⁶³)NH₂, —N₃, FL, or Bm;
each of a and b is independently an integer selected from the range of 1 to 100;
each n is independently an integer selected from the range of 1 to 10;
each of e, f, g, and h is independently 0 or 1;
each of R¹¹-R³³ is independently hydrogen, C₁-C₂₀ alkyl, or C₅-C₂₀ aryl;
each of R⁴⁰-R⁶¹ is independently hydrogen or C₁-C₁₀ alkyl;
each of R⁶² and R⁶³ is independently a side chain residue of a natural α-amino acid;
each FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an irnidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound;
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent, wherein the detectable agent is differentially separated from the bodily fluid by the organ or tissue;
exposing the detectable agent in the bodily fluid to electromagnetic radiation for exciting emission from the detectable agent;
measuring the emission from the detectable agent that is in the bodily fluid; and
determining the physiological function of the organ or tissue of the subject based on measurement of the emission.

60. The method of claim 59 wherein the organ or tissue is a kidney, or tissue or cells thereof, of the subject; or wherein the organ or tissue is a liver, or tissue or cells thereof, of the subject.

61. A compound being of the formula (FX2): 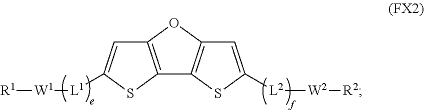
wherein:
each of L¹ and L², if present, is independently C₁-C₁₀ alkylene, C₃-C₁₀ cycloalkylene, C₂-C₁₀ alkenylene, C₃-C₁₀ cycloalkenylene, C₂-C₁₀ alkynylene, ethenylene, ethynylene, phenylene, 1-aza-2,5-dioxocyclopentylene, 1,4-diazacyclohexylene, —(CH₂CH₂O)_{b}—, or —(CHOH)ₐ—;
W¹ is a single bond, —SO—, —SO₂—, or —CO—;
W² a single bond, —(CH₂)ₙ—, —(HCCH)ₙ—, —O—, —S—, —SO—, —SO₂—, —SO₃—, —OSO₂—, —NR¹¹—, —CO—, —COO—, —OCO—, —OCOO—, —CONR¹²—, —NR¹³CO—, —OCONR¹⁴—, —NR¹⁵COO—, —NR¹⁶CONR¹⁷—, —NR¹⁸CSNR¹⁹—, —O(CH₂)ₙ—, —S(CH₂)ₙ—, —NR²⁰(CH₂)ₙ—, —CO(CH₂)ₙ—, —COO(CH₂)ₙ—, —OCO(CH₂)ₙ—, —OCOO(CH₂)ₙ—, —CONR²¹(CH₂)ₙ—, —CONR²²(CH₂)ₙ—, —NR²³CO(CH₂)ₙ—, —OCONR²⁴(CH₂)ₙ—, —NR²⁵COO(CH₂)ₙ—, NR²⁶CONR²⁷(CH₂)ₙ—, —NR²⁸CSNR²⁹(CH₂)ₙ—, —O(CH₂)ₙNR³⁰CO(CH₂)ₙ—, —CO(CH₂)ₙ(CH₂OCH₂)ₙ(CH₂)ₙNR³¹(CH₂)ₙNR³²CO—, or —CO(CH₂)ₙNR³³CO—;
R¹ is —N₃, —SOR⁴¹, or —OSR⁴²;
R² is a hydrogen, —OCF₃, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, C₁-C₆ alkoxycarbonyl, halo, halomethyl, dihalomethyl, trihalomethyl, —CO₂R⁴⁰, —SOR⁴¹, —OSR⁴², —SO₂OR⁴³, —CH₂(CH₂OCH₂)_{c}CH₂OH, —PO₃R⁴⁴R⁴⁵, —OR⁴⁶, —SR⁴⁷, —NR⁴⁸R⁴⁹, —NR⁵⁰COR⁵¹, —CN, —CONR⁵²R⁵³, —COR⁵⁴, —NO₂, —SO₂R⁵⁵, —PO³R⁵⁶R⁵⁷, —SO₂NR⁵⁸R⁵⁹, —CH₂(CHOH)ₐR⁶⁰, —(CH₂CH₂O)_{b}R⁶¹, —CH(R⁶²)CO₂H, —CH(R⁶³)NH₂, —N₃, PS¹, PS², FL, or Bm;
each of a and b is independently an integer selected from the range of 1 to 100;
each n is independently an integer selected from the range of 1 to 10;
each of e and f is independently 0 or 1;
each of R¹¹-R³³ is independently hydrogen, C₁-C₂₀ alkyl, or C₅-C₂₀ aryl;
each of R⁴⁰-R⁶¹ is independently hydrogen or C₁-C₁₀ alkyl;
each of R⁶² and R⁶³ is independently a side chain residue of a natural α-amino acid;
each FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthene, a flavone, a coumarin, a phenoxazine, a phenothiazine, a phenoselenazine, a cyanine, an indocyanine, or an azo compound;
each PS¹ is independently a Type 1 photosensitizer;
each PS² is independently a Type 2 photosensitizer; and
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent.

62. The compound of claim 61, wherein at least one of R¹ and R² is Bm.

63. The compound of claim 61, wherein at least one of R^{I} and R² is —OR⁴⁶, —SR⁴⁷, —NR⁴⁸R⁴⁹, or —NR⁵⁰COR⁵¹.

64. The compound of claim 61, wherein at least one of R¹ and R² is —CN, —CO₂R⁴⁰, —COR⁵⁴, —NO₂, —SO₂R⁵⁵, or 13 SO₂NR⁵⁸R⁵⁹.

65. The compound of claim 61, wherein at least one of R¹ and R² is —NR⁴⁸R⁴⁹, and at least one of R¹ and R² is —CO₂R⁴⁰, —COR⁵⁴, —SO₂NR⁵⁸R⁵⁹, or —SO₂R⁵⁵.

66. The compound of claim 61, wherein at least one of R¹ and R² is PS¹, and each PS¹ is independently an azide, azo, diazo, oxaza, or diaza group.

67. The compound of claim 61, wherein at least one of R¹ and R² is PS², wherein each PS² is independently a porphyrin, benzoporphyrin, phthalocyanine, phenothiazine, chlorin, bacteriochlorin, purpurin, merocyanine, pheophorbides, psoralen, aminolevulinic acid (ALA), hematoporphyrin derivative, porphycene, porphacyanine, cyanine, indocyanine, rhodamine, phenoxazine, a phenoselenazine, fluorescein, squaraine, corrin, croconium, azo dye, methine dye, indolenium dye, halogen, anthracyline, C₁-C₂₀ peroxyalkyl, C₁-C₂₀ peroxyaryl, C₁-C₂₀ sulfenatoalkyl, sulfenatoaryl, naphthalocyanine, methylene blue, or chalcogenopyrylium analogue.

68. A compound being of the formula (FX3): 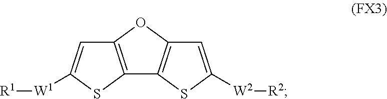
wherein:
W¹ is —O—, —S—, —NR¹¹—, —OCO—, —OCOO—, —NR¹³CO—, —CONR¹²—, —OCONR¹⁴, or —NR¹⁵COO—;
W² is —SO—, —SO₂—, —SO₃—, —COO—, or —CONR¹²—;
R¹ is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL, or Bm; and
R² is hydrogen, C₁-C₂₀ alkyl, C₅-C₂₀ aryl, C₅-C₂₀ alkylaryl, —CH₂(CHOH)ₐR⁶⁰, —(CH₂CH₂O)_{b}R⁶¹, —CH(R⁶²)CO₂H, —CH(R⁶³)NH₂, PS¹, PS², FL, or Bm.
each of a and b is independently an integer selected from the range of 1 to 100;
each of R¹¹-R¹⁵ is independently hydrogen, C₁-C₂₀ alkyl, or C₅-C₂₀ aryl;
each of R⁶⁰ and R⁶¹ is independently hydrogen or C₁-C₁₀ alkyl;
each of R⁶² and R⁶³ is independently a side chain residue of a natural α-amino acid;
each FL is independently a fluorescent group corresponding to a naphthoquinone, an anthracene, an anthraquinone, a phenanthrene, a tetracene, a naphthacenedione, a pyridine, a quinoline, an isoquinoline, an indole, an isoindole, a pyrrole, an imidiazole, a pyrazole, a pyrazine, a purine, a benzimidazole, a benzofuran, a dibenzofuran, a carbazole, an acridine, an acridone, a phenanthridine, a thiophene, a benzothiophene, a dibenzothiophene, a xanthene, a xanthone, a flavone, a coumarin, a phenoxazine, a phenothiazine, a phenoselenazine, a cyanine, an indocyanine, or an azo compound;
each PS¹ is independently a Type 1 photosensitizer;
each PS² is independently a Type 2 photosensitizer; and
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a drug mimic, a hormone, a receptor, a metal chelating agent, a radioactive or nonradioactive metal complex, a mono- or polynucleotide comprising 1 to 50 nucleic acid units, a polypeptide comprising 2 to 30 amino acid units, or an echogenic agent.

69. The compound of claim 68, wherein W¹ is —NR¹¹— or —CONR¹²—, and W² is —COO— or —CONR¹²—.
